(19) United States
(12) Patent Application Publication
(10) Pub. No.: US 2019/0091339 A1
(43) Pub. Date: Mar. 28, 2019

(54) ABIRATERONE-CYCLIC OLIGOMER PHARMACEUTICAL FORMULATIONS AND METHODS OF FORMATION AND ADMINISTRATION THEREOF

(71) Applicant: DISPERSOL TECHNOLOGIES, LLC, Georgetown, TX (US)

(72) Inventors: Dave Miller, Round Rock, TX (US); Urvi Gala, Austin, TX (US)

(73) Assignee: DISPERSOL TECHNOLOGIES, LLC, Georgetown, TX (US)

(21) Appl. No.: 16/136,413

(22) Filed: Sep. 20, 2018

Related U.S. Application Data
(60) Provisional application No. 62/562,081, filed on Sep. 22, 2017.

(51) Int. Cl. A61K 47/40; A61K 31/58; A61K 9/10; A61K 45/06; A61K 9/20; A61P 35/00

(52) U.S. Cl. CPC A61K 47/40; A61K 31/58; A61K 9/10; A61P 35/00; A61K 9/2059; A61K 9/2054; A61K 45/06

(57) ABSTRACT

The present disclosure relates to pharmaceutical formulations including abiraterone and a cyclic oligomer, as well as tablets including such pharmaceutical formulations, methods of forming such pharmaceutical formulations, and methods of administering such pharmaceutical formulations or tablets.

### PRIORITY CLAIM

This application claims benefit of priority to U.S. Provisional Application Ser. No. 62/562,081, filed Sep. 22, 2017, the entire contents of which are hereby incorporated by reference.

### TECHNICAL FIELD

The present disclosure relates to abiraterone pharmaceutical formulations and methods of forming and administering such pharmaceutical formulations.

### BACKGROUND

Certain types of advanced prostate cancer are often difficult to treat because cancer cell growth is driven by androgens. Androgens are made primarily by the testes in adult males, but they are also produced by the adrenal glands and, in the case of some prostate cancers, by the cancer cells themselves. As a result, some advanced prostate cancers continue to exhibit androgen-induced growth even after castration of the patient. Abiraterone blocks androgen production, and particular testosterone production in the testes, adrenal glands, and cancer cells themselves. Accordingly, orally administered abiraterone acetate has been approved for use in patients with metastatic castration-resistant prostate cancer (mCRPC). In addition, abiraterone has shown potential efficacy in the treatment of other androgen sensitive cancers, e.g., breast cancer.

Abiraterone blocks androgen biosynthesis by inhibiting Cytochrome P450 17A1 (CYP17A1). As a result, patients taking abiraterone may experience general negative effects of insufficient glucocorticoid levels, such as low serum cortisol and a compensatory increase in adrenocorticotropic hormone. Patients taking abiraterone are, therefore, typically also given glucocorticoid replacement therapy.

Abiraterone is highly lipophilic and has low aqueous solubility in the gastrointestinal tract, thus severely limiting the drug's oral bioavailability. The leading commercial product, Zytiga, mitigates this insolubility issue by use of the more soluble ester prodrug, abiraterone acetate. However, the effectiveness of the prodrug toward improving bioavailability is limited, as evidenced by the food effect and pharmacokinetic variability cited in the label. Specifically, the 10-fold increase in AUC when Zytiga is administered with a high fat meal suggests that the absolute bioavailability of abiraterone is maximally 10% when Zytiga is administered per the label (fasted). Further, exposure was not significantly increased when the Zytiga dose was doubled from 1,000 to 2,000 mg (8% increase in the mean AUC). The results of this study imply that Zytiga is dosed near the absorption limit.

In the treatment of metastatic castration resistant prostate cancer with abiraterone, reductions in prostate specific antigen (PSA) are predictive of improved clinical outcomes. In well controlled trials, dosing with 1,000 mg abiraterone acetate daily only achieves target PSA reductions in up to 60% of treated patients. Thus, abiraterone remains a difficult drug to administer optimally.

Additionally, recent findings have suggested that abiraterone response in patients with metastatic castration-resistant prostate cancer is correlated to steady state trough levels (Cmin). (Xu et al., Clin. Pharmacokinet. 56: 55-63, 2017) Specifically, Cmin values greater than about 30 ng/mL correlate with greater PSA decay rate, suggesting that improved abiraterone bioavailability and optimizing the pharmacokinetic profile would lead to better therapeutic efficacy, i.e., anti-tumor response. This finding indicates that the therapeutic benefit of Zytiga is limited by suboptimal abiraterone delivery and highlights a critical need for improved abiraterone compositions, specifically those that can increase systemic abiraterone exposure and trough levels.

### SUMMARY

The present disclosure provides a pharmaceutical formulation including abiraterone and a cyclic oligomer excipient.

According to various further embodiments of the pharmaceutical formulation, which may all be combined with one another unless clearly mutually exclusive:

i) the abiraterone and cyclic oligomer excipient may be in an amorphous solid dispersion;

i-a) the amorphous solid dispersion may contain less than 5% crystalline material, less than 1% crystalline material, or no crystalline material;

ii) the abiraterone may include at least 99% abiraterone;

iii) the abiraterone may include at least 99% abiraterone, having the structural formula:

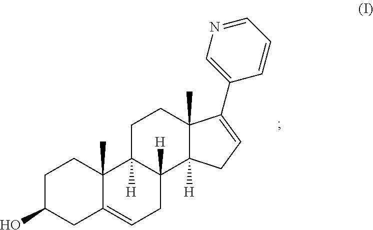

iv) the abiraterone may include at least 99% abiraterone salt;

v) the abiraterone may include at least 99% abiraterone ester;

v-a) the abiraterone ester may include abiraterone acetate, having the structural formula:

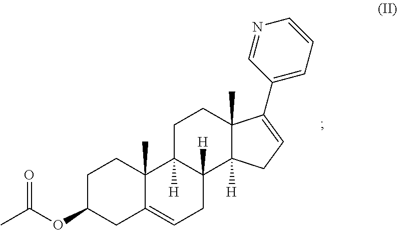

vi) the abiraterone may include at least 99% abiraterone solvate;

vii) the abiraterone may include at least 99% abiraterone hydrate;

viii) the pharmaceutical formulation may include 10 mg, 25 mg, 50 mg, 70 mg, 100 mg, or 250 mg of amorphous abiraterone;

ix) the pharmaceutical formulation may include an amount of amorphous abiraterone sufficient to achieve the same or greater therapeutic effect, bioavailability, Cₘᵢₙ, Cₘₐₓ or Tₘₐₓ in a patient as 50 mg, 70 mg, 100 mg, 250 mg, 500 mg, or 1000 mg of crystalline abiraterone or crystalline abiraterone acetate when consumed on an empty stomach;

x) the pharmaceutical formulation may include comprising 10 mg, 25 mg, 50 mg, 70 mg, 100 mg, 250 mg or 500 mg of amorphous abiraterone;

xi) the pharmaceutical formulation may include an amount of amorphous abiraterone sufficient to achieve the same or greater therapeutic effect, bioavailability, Cₘᵢₙ, Cₘₐₓ or Tₘₐₓ in a patient as 10 mg, 25 mg, 50 mg, 70 mg, 100 mg, 250 mg, 500 mg or 1000 mg of crystalline abiraterone or crystalline abiraterone acetate when consumed on an empty stomach;

xii) the pharmaceutical formulation may include 1,000 mg of amorphous abiraterone;

xiii) the pharmaceutical formulation may include an amount of amorphous abiraterone sufficient to achieve the same or greater therapeutic effect, bioavailability, Cₘᵢₙ, Cₘₐₓ or Tₘₐₓ in a patient as 1,000 mg of crystalline abiraterone or crystalline abiraterone acetate when consumed on an empty stomach;

xiv) the abiraterone and cyclic oligomer may be present in a molar ratio of 1:0.25 to 1:25;

xv) the abiraterone and cyclic oligomer may be present in a molar ratio of at least 1:2;

xvi) the amorphous solid dispersion may include 1% to 50% by weight abiraterone;

xvii) the amorphous solid dispersion may include at least 10% by weight abiraterone;

xviii) the cyclic oligomer excipient may include a cyclic oligosaccharide or cyclic oligosaccharide derivative;

xvii-a) the cyclic oligosaccharide or cyclic oligosaccharide derivative may include a cyclodextrin or a cyclodextrin derivative;

xvii-a-a) the cyclodextrin derivative may include a hydroxy propyl β cyclodextrin;

xvii-a-b) the cyclodextrin derivative may include a sodium (Na) sulfo-butyl ether β cyclodextrin;

xvii-a-c) the cyclodextrin derivative may include a hydroxypropyl group

xvii-a-d) the cyclodextrin derivative may include a sulfo-butyl ether functional group;

xvii-a-e) the cyclodextrin derivative may include a methyl group;

xvii-a-f) the cyclodextrin derivative may include a carboxymethyl group;

xix) the amorphous solid dispersion may include 50% to 99% by weight cyclic oligomer excipient;

xx) the amorphous solid dispersion may include at least 90% by weight cyclic oligomer excipient;

xxi) the amorphous solid dispersion may include an additional excipient;

xxi-a) the cyclic oligomer excipient may be a primary excipient;

xxi-b) the additional excipient may be the primary excipient;

xxi-b-a) the additional excipient may be a secondary excipient;

xxi-c) the additional excipient may be a polymer excipient;

xxi-c-a) the polymer excipient may be water soluble;

xxi-c-b) the polymer excipient may include a non-ionic polymer;

xxi-c-c) the polymer excipient may include an ionic polymer;

xxi-c-d) the polymer excipient may include a hydroxy propyl methyl cellulose acetate succinate;

xxi-c-d-a) the hydroxypropylmethyl cellulose acetate succinate may have 5-14% acetate substitution and 4-18% succinate substitution;

xxi-c-d-a-a) the hydroxypropylmethyl cellulose acetate succinate may have 10-14% acetate substitution and 4-8% succinate substitution;

xxi-c-d-a-a-a) the hydroxypropylmethyl cellulose acetate succinate may have 12% acetate substitution and 6% succinate substitution;

xxi-d) the amorphous solid dispersion may include between 1% and 49% by weight additional excipient;

xxi-e) the amorphous solid dispersion may include 10% by weight or less additional excipient;

xxii) the pharmaceutical formulation may include a glucocorticoid replacement API;

xxii-a) the glucocorticoid replacement API may include prednisone, methylprednisone, prednisolone, methylprednisolone, dexamethasone, or a combination thereof;

The disclosure further provides a tablet for oral administration, which may include any pharmaceutical formulation above or otherwise described herein.

According to various further embodiments of the tablet, which may all be combined with one another unless clearly mutually exclusive:

i) the tablet may include a coating;

i-a) the coating may include a glucocorticoid replacement API;

i-a-a) the glucocorticoid replacement API may include prednisone, methylprednisone, prednisolone, methylprednisolone, dexamethasone, or a combination thereof;

ii) the tablet may include an external phase including an additional amount of the cyclic oligomer excipient;

iii) the tablet may include an external phase including at least one additional excipient;

iv) the tablet may include a concentration enhancing polymer;

iv-a) the concentration enhancing polymer may include a hydroxypropylmethyl cellulose acetate succinate.

v) the tablet may include an external phase including one or more water swellable polymers

v-a) the water swellable polymers may include polyethylene oxide, hydroxymethyl cellulose, hydroxyethyl cellulose, hydroxypropyl cellulose, hydroxypropylmethyl cellulose, and carboxymethyl cellulose

v-b) the tablet may be of a geometry such that when the water swellable polymers are hydrated the size and shape of the tablet prevents passage of the tablet through the pylorus of the stomach

v-c) the tablet may have drug release profile such as immediate release, or modified release such as extended release which may be sustained release or controlled release, or pulsatile release or delayed release

The tablet may comprise an external phase comprising at least one additional drug release modifying excipient, or may comprise an external phase comprising of one or more hydrogel forming excipient, or may comprise an external phase comprising of combination of polyethylene oxide and hydroxypropyl methyl cellulose.

The present disclosure also provides a method of forming a pharmaceutical formulation by compounding crystalline abiraterone and a cyclic oligomer excipient in a thermokinetic mixer at a temperature less than or equal to 200° C. for less than 300 seconds to form an amorphous solid dispersion of abiraterone and cyclic oligomer excipient.

According to various further embodiments of the method, which may all be combined with one another unless clearly mutually exclusive:

i) the pharmaceutical formulation may be any pharmaceutical formulation above or otherwise described herein;

ii) the method may also include compounding at least one additional excipient with the crystalline abiraterone and cyclic oligomer excipient to form the solid amorphous dispersion;

iii) compounding in the thermokinetic mixer may not cause substantial thermal degradation of the abiraterone;

iv) compounding in the thermokinetic mixer may not cause substantial thermal degradation of the cyclic oligomer excipient;

v) compounding in the thermokinetic mixer may not cause substantial thermal degradation of the additional excipient.

The present disclosure also provides a method of forming a pharmaceutical formulation, by melt processing crystalline abiraterone and a cyclic oligomer excipient to form an amorphous solid dispersion of abiraterone and the cyclic oligomer excipient in which the abiraterone is not substantially thermally degraded.

According to various further embodiments of the method, which may all be combined with one another unless clearly mutually exclusive:

i) the pharmaceutical formulation may be any pharmaceutical formulation above or otherwise described herein;

ii) the method may also include processing at least one additional excipient with the crystalline abiraterone and cyclic oligomer excipient to form the solid amorphous dispersion;

iii) melt processing may not cause substantial thermal degradation of the cyclic oligomer excipient;

iv) melt processing may not cause substantial thermal degradation of the additional excipient.

The present disclosure further provides a method of forming a pharmaceutical formulation by comprising dissolving crystalline abiraterone and a cyclic oligomer excipient in a common organic solvent to form a dissolved mixture and spray drying the dissolved mixture to form an amorphous solid dispersion of abiraterone and cyclic oligomer excipient.

According to various further embodiments of the method, which may all be combined with one another unless clearly mutually exclusive:

i) the pharmaceutical formulation may be any pharmaceutical formulation above or otherwise described herein;

ii) the method may further include dissolving at least one additional excipient with the crystalline abiraterone and cyclic oligomer excipient and spray drying to form the solid amorphous dispersion;

iii) spray drying may not cause substantial thermal degradation of the abiraterone;

iv) spray drying may not cause substantial thermal degradation of the cyclic oligomer excipient;

v) spray drying may not cause substantial thermal degradation of the additional excipient.

The present disclosure also includes any pharmaceutical formulations prepared according to any of the above methods, which may also have any of the other features of pharmaceutical formulations described above or otherwise herein.

The present disclosure also includes tablets containing any pharmaceutical formulations prepared according to any of the above methods, which may also have any of the other features of pharmaceutical formulations or tablets described above or otherwise herein.

The present disclosure also provides a method of treating prostate cancer in a patient by administering any pharmaceutical formulation described above or otherwise herein or any tablet described above or otherwise herein to a patient having prostate cancer.

According to various further embodiments of the method, which may all be combined with one another unless clearly mutually exclusive:

i) the patient may have castration-resistant prostate cancer, metastatic castration-resistant prostate cancer, metastatic prostate cancer, locally advanced prostate cancer, relapsed prostate cancer, or other high-risk prostate cancer;

ii) the patient may have previously received treatment with chemotherapy;

ii-a) the chemotherapy may include docetaxel;

iii) the patient may have previously received treatment with enzalutamide;

iv) the patient may have previously experienced a sub-optimal response to crystalline abiraterone acetate;

v) the pharmaceutical formulation or tablet may be administered to the patient in combination with androgen-deprivation therapy;

vi) the pharmaceutical formulation or tablet may be administered to the patient in combination with a glucocorticoid replacement API;

vii) the pharmaceutical formulation or tablet may be administered once daily;

viii) the pharmaceutical formulation or tablet may be administered twice daily;

ix) the pharmaceutical formulation or tablet may include amorphous abiraterone and may be administered at dose lower in weight of abiraterone as compared to a dose in weight of abiraterone acetate sufficient to achieve an equivalent or higher therapeutic effect, bioavailability, Cₘᵢₙ, Cₘₐₓ or Tₘₐₓ.

The present disclosure also provides a method of treating various androgen sensitive cancers by administering any pharmaceutical formulation described above or otherwise herein or any tablet described above or otherwise herein to a patient having an androgen sensitive cancer.

According to various further embodiments of the method, which may all be combined with one another unless clearly mutually exclusive:
i. the patient may have breast cancer or triple-negative androgen receptor positive locally advanced/metastatic breast cancer or ER-positive HER2-negative breast cancer or ER positive metastatic breast cancer or apocrine breast cancer;
ii. the patient may have Cushing's syndrome with adrenocortical carcinoma;
iii. the patient may have urothelial carcinoma or bladder cancer or urinary bladder neoplasms;
iv. the patient may have androgen receptor expressing, relapsed/metastatic, salivary gland cancer or recurrent and/or metastatic salivary gland cancer or salivary glands tumors or salivary duct carcinoma;
v. the patient may have previously received treatment with chemotherapy;
   iv-a) the chemotherapy may include docetaxel;
vi. the patient may have previously received treatment with medication used for breast cancer, adrenal carcinoma and salivary gland cancer;
vii. the patient may have previously experienced a sub-optimal response to crystalline abiraterone acetate;
viii. the pharmaceutical formulation or tablet may be administered to the patient in combination with androgen-deprivation therapy;
ix. the pharmaceutical formulation or tablet may be administered to the patient in combination with a glucocorticoid replacement API;
x. the pharmaceutical formulation or tablet may be administered once daily;
xi. the pharmaceutical formulation or tablet may be administered twice daily;
xii. the pharmaceutical formulation or tablet may include amorphous abiraterone and may be administered at dose lower in weight of abiraterone as compared to a dose in weight of abiraterone acetate sufficient to achieve an equivalent or higher therapeutic effect, bioavailability, Cₘᵢₙ, Cₘₐₓ or Tₘₐₓ.

It is contemplated that any method or composition described herein can be implemented with respect to any other method or composition described herein.

It is contemplated that any embodiment discussed in this specification can be implemented with respect to any method or composition of the invention, and vice versa. Furthermore, compositions and kits of the invention can be used to achieve methods of the invention.

Throughout this application, the term “about” is used to indicate that a value includes the inherent variation of error for the device, the method being employed to determine the value, or the variation that exists among the study subjects.

Unless it is otherwise clear that a single entity is intended, terms such as “a,” “an,” and “the” are not intended to refer to only a singular entity and include the general class of which a specific example is described for illustration.

In addition, unless it is clear that a precise value is intended, numbers recited herein should be interpreted to include variations above and below that number that may achieve substantially the same results as that number, or variations that are “about” the same number.

Finally, a derivative of the present disclosure may include a chemically modified molecule that has an addition, removal, or substitution of a chemical moiety of the parent molecule.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure may be further understood through reference to the attached figures in combination with the detailed description that follows.

FIG. 1 is an X-ray diffractogram of neat crystalline abiraterone.

FIG. 2 is a set of X-ray diffractograms of abiraterone solid dispersions with various polymer excipients. Excipient type (cellulose-based, polyvinyl-based, or acrylate-based) is indicated.

FIG. 3 is an X-ray diffractogram of an amorphous solid dispersion of abiraterone and hydroxy propyl β cyclodextrin.

FIG. 4 is a graph of concentration of dissolved abiraterone versus time (dissolution profile) for neat crystalline abiraterone or various solid dispersions of abiraterone with a polymer excipient or a hydroxy propyl β cyclodextrin excipient.

FIG. 5 is a graph of concentration of dissolved abiraterone versus time (dissolution profile) for amorphous solid dispersions of abiraterone with a hydroxy propyl β cyclodextrin primary excipient in the presence of various polymer secondary excipients. Only the neutral phase dissolution profile is shown.

FIG. 6 is a set of X-ray diffractograms of amorphous solid dispersions of abiraterone with various amounts of a hydroxy propyl β cyclodextrin primary excipient, and various amounts of a hydroxy propyl methyl cellulose acetate succinate with 10-14% acetate substitution and 4-8% of succinate substitution as the secondary excipient.

FIG. 7 is a set of graphs of concentration of dissolved abiraterone versus time (dissolution profile) for amorphous solid dispersions of abiraterone with various amounts of a hydroxy propyl β cyclodextrin primary excipient, and various amounts of a hydroxy propyl methyl cellulose acetate succinate with 10-14% acetate substitution and 4-8% of succinate substitution as a secondary excipient. Upper panel (A) provides a dissolution profile in the acidic phase, while lower panel (B) provides a dissolution profile in the neutral phase.

FIG. 8 is a graph of concentration of dissolved abiraterone versus time (dissolution profile) as a function of the amount of drug loaded into the dissolution vessel (25 to 200 times the intrinsic solubility) for amorphous solid dispersions of abiraterone with a polymer excipient or a hydroxy propyl β cyclodextrin primary excipient and hydroxy propyl methyl cellulose acetate succinate with 10-14% acetate substitution and 4-8% of succinate substitution as a secondary excipient.

FIG. 9 is an X-ray diffractogram of amorphous solid dispersions of abiraterone and hydroxy propyl β cyclodextrin in 1:4 (Example 7.1) and 3:7 (Example 7.2) weight ratios formed by thermokinetic processing.

FIG. 10 is a graph of concentration of dissolved abiraterone versus time (dissolution profile) for solid dispersions of abiraterone and hydroxy propyl β cyclodextrin in weight ratios of 1:9 (Example 2.4), 1:4 (Example 7.1), and 3:7 (Example 7.2) formed by thermokinetic compounding.

FIG. 11. X-ray diffractogram of neat crystalline abiraterone acetate.

FIG. 12 is a set of X-ray diffractograms of abiraterone acetate solid dispersions with various polymer excipients. Excipient type (cellulose-based, polyvinyl-based, or acrylate-based) is indicated.

FIG. 13 is a graph of concentration of dissolved abiraterone acetate versus time (dissolution profile) for neat crystalline abiraterone acetate or various solid dispersions of abiraterone acetate with a polymer excipient.

FIG. 14 is an X-ray diffractogram of an amorphous solid dispersion of abiraterone acetate and hydroxy propyl β cyclodextrin.

FIG. 15 is a graph of concentration of dissolved abiraterone acetate versus time (dissolution profile) for neat crystalline abiraterone acetate and an amorphous solid dispersion of abiraterone acetate with hydroxy propyl β cyclodextrin.

FIG. 16 is an X-ray diffractogram of amorphous solid dispersions of abiraterone acetate and hydroxy propyl β cyclodextrin in 1:4 (Example 10.1) weight ratio formed by thermokinetic processing.

FIG. 17 is a graph of concentration of dissolved abiraterone acetate versus time (dissolution profile) for solid dispersions of abiraterone acetate and hydroxy propyl β cyclodextrin in weight ratios of 1:9 (Example 9.1) and 1:4 (Example 10.1)

FIG. 18 is a graph of concentration of dissolved abiraterone acetate versus time (dissolution profile) as a function of the amount of drug loaded into the dissolution vessel (100 to 400 times the intrinsic solubility) in the form of an abiraterone acetate-hydroxy propyl β cyclodextrin (1:9 w/w) ASD.

FIG. 19. Graph of abiraterone concentration versus time from dissolution testing of 50 mg tablets made per Example 10 in 900 ml of 0.01 N HCl.

FIG. 20. Abiraterone plasma concentration versus time profiles following oral administration to male beagle dogs of abiraterone IR and XR tablets (50 mg abiraterone) made per Examples 11.1 and 11.2, respectively, relative to the reference, Zytiga (250 mg abiraterone acetate).

FIG. 21. Total oral abiraterone exposure (AUC) versus dose curve from an ascending dose PK study in SCID mice comparing the composition made per Example 2.4 versus abiraterone acetate.

FIG. 22. Tumor growth curves following once-daily administration of abiraterone acetate or the composition from Example 2.4 at two dose levels to 22RV1 xenograft mice

### DETAILED DESCRIPTION

The present disclosure relates to abiraterone pharmaceutical formulations and methods of forming and administering such pharmaceutical formulations.

### A. Pharmaceutical Formulation

A pharmaceutical formulation of the present disclosure may include abiraterone as an active pharmaceutical ingredient (API). Abiraterone, unless otherwise specified herein, includes both the active form of abiraterone and its modified forms, in either amorphous or crystalline states. Modified forms of abiraterone include a pharmaceutically acceptable salt, ester, derivative, analog, prodrug, hydrate, or solvate thereof.

Abiraterone is (3β)-17(3-pyridinyl)androsta-5,16-dien-3-ol and has the formula:

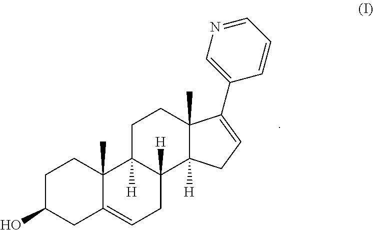

Abiraterone acetate, such as ZYTIGA®, is an ester of abiraterone, (3β)-17(3-pyridinyl)androsta-5,16-dien-3-ol acetate, and has the formula:

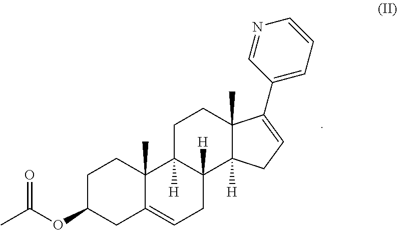

The pharmaceutical formulation may include abiraterone, which, prior to the present disclosure, has proven resistant to pharmaceutical formulation with sufficient bioavailability or therapeutic effect. In particular, to the extent a pharmaceutical formulation of the present disclosure includes both abiraterone and in a modified form, such as a pharmaceutically acceptable salt, ester, derivative, analog, prodrug, hydrate, or solvate thereof, the pharmaceutical formulation may include at least 80%, at least 95%, at least 99%, or at least 99% abiraterone as compared to total abiraterone and modified abiraterone by molecular percentage, by weight, or by volume.

The abiraterone in a pharmaceutical formulation of the present disclosure may lack substantial impurities. For example, the abiraterone may lack impurities at levels beyond the threshold that has been qualified by toxicology studies, or beyond the allowable threshold for unknown impurities as established in the Guidance for Industry, Q3B(R2) Impurities in New Drug Products (International Committee for Harmonization, published by the U.S. Department of Health and Human Services, Food and Drug Administration, Center for Drug Evaluation and Research (CDER), Center for Biologics Evaluation and Research, July, 2006, incorporated by reference herein. Alternatively, the abiraterone in a pharmaceutical formulation of the present disclosure may have less than 1.0%, 0.75%, 0.5%, 0.1%, 0.05%, or 0.01% impurities by weight as compared to total weight of abiraterone and impurities, relative to a standard of known concentration in mg/mL. As another alternative, the abiraterone in a pharmaceutical formulation of the present disclosure may retain at least 90%, 91%, 92%, 93%, 94%, 95%, 96%, 97%, 98%, 99%, or 99.9% drug activity or potency as compared to the uncompounded abiraterone as measured by HPLC. Impurities may include abiraterone degradation products, such as thermal degradation products.

In specific examples, a pharmaceutical formulation of the present disclosure including abiraterone may further include a glucocorticoid replacement API. Suitable glucocorticoid replacement APIs may have an intermediate biological half-life, such as between 18 and 36 hours, or a long biological half-life, such as between 36 and 54 hours. Suitable glucocorticoid APIs include dexamethasone, prednisone or prednisolone or alkylated forms, such as methyl prednisone and methyl prednisolone, and any combinations thereof. Other glucocorticoid replacement APIs may also be used.

The glucocorticoid replacement API in a pharmaceutical formulation of the present disclosure may also not contain substantial levels of impurities. For example, the glucocorticoid replacement may not have impurities at levels beyond the threshold that has been qualified by toxicology studies, or beyond the allowable threshold for unknown impurities as established in the Guidance for Industry, Q3B(R2) Impurities in New Drug Products (International Committee for Harmonization, published by the U.S. Department of Health and Human Services, Food and Drug Administration, Center for Drug Evaluation and Research (CDER), Center for Biologics Evaluation and Research, July, 2006, incorporated by reference herein. Alternatively, the glucocorticoid replacement API in a pharmaceutical formulation of the present disclosure may be have less than 1.0%, 0.75%, 0.5%, 0.1%, 0.05%, or 0.01% impurities by weight as compared to total weight of glucocorticoid replacement API and impurities, relative to a standard of known concentration in mg/mL. As another alternative, the glucocorticoid replacement API in a pharmaceutical formulation of the present disclosure may retain at least 90%, 91%, 92%, 93%, 94%, 95%, 96%, 97%, 98%, 99%, or 99.9% drug activity or potency as compared to the uncompounded glucocorticoid replacement API as measured by HPLC. Impurities may include glucocorticoid replacement API degradation products, such as thermal degradation products.

A pharmaceutical formulation of the present disclosure may further include one or more other APIs in addition to abiraterone. Suitable additional APIs include other APIs approved to treat prostate cancer, or a side effect of prostate cancer or prostate cancer treatment. These additional APIs may be in their active form. These APIs may be compoundable even when they have not been previously compoundable, compoundable in an orally administrable pharmaceutical formulation, compoundable with abiraterone, or compoundable in their active forms. Suitable additional APIs include those used in androgen-deprivation therapy, non-steroidal androgen receptor inhibitors, taxanes, gonadotrophin-releasing hormone antagonists, gonadotropin-releasing hormone analogs, androgen receptor antagonists, non-steroidal anti-androgens, analogs of luteinizing hormone-releasing hormone, anthracenedione antibiotics, and radiopharmaceuticals, and any combinations thereof. These suitable additional APIs include apalutamide, such as ERLEADA™ (Janssen), bicalutamide, such as CASODEX® (AstraZenica, North Carolina, US), cabazitaxel, such as JEVTANA® (Sanofi-Aventis, France), degarelix, docetaxel, such as TAXOTERE® (Sanofi-Aventis), enzalutamide, such as XTANDI® (Astellas Pharma, Japan), flutamide, goserelin acetate, such as ZOLADEX® (TerSera Therapeutics, Iowa, US), leuprolide acetate, such as LUPRON® (Abbvie, Ill., US), LUPRON® DEPOT (Abbive), LUPRON® DEPOT-PED (Abbive), and VIADUR® (ALZA Corporation, California, US), mitoxantrone hydrochloride, nilutamide, such as NILANDRON® (Concordia Pharmaceuticals, Barbados), and radium 223 dichloride, such a XOFIGO® (Bayer Healthcare Pharmaceuticals, New Jersey, US), and any combinations thereof.

Any additional API in a pharmaceutical formulation of the present disclosure may also not contain substantial levels of impurities. For example, the additional API may not have impurities at levels beyond the threshold that has been qualified by toxicology studies, or beyond the allowable threshold for unknown impurities as established in the Guidance for Industry, Q3B(R2) Impurities in New Drug Products (International Committee for Harmonization, published by the U.S. Department of Health and Human Services, Food and Drug Administration, Center for Drug Evaluation and Research (CDER), Center for Biologics Evaluation and Research, July, 2006, incorporated by reference herein. Alternatively, the additional API in a pharmaceutical formulation of the present disclosure may be have less than 1.0%, 0.75%, 0.5%, 0.1%, 0.05%, or 0.01% impurities by weight as compared to total weight of additional API and impurities, relative to a standard of known concentration in mg/mL. As another alternative, the additional API in a pharmaceutical formulation of the present disclosure may retain at least 90%, 91%, 92%, 93%, 94%, 95%, 96%, 97%, 98%, 99%, or 99.9% drug activity or potency as compared to the uncompounded additional API as measured by HPLC. Impurities may include additional API degradation products, such as thermal degradation products.

A pharmaceutical formulation of the present disclosure further includes at least one excipient. When multiple excipients are used in a pharmaceutical formulation of the present disclosure, the one present in the largest amount by weight percent is typically referred to as the primary excipient, with other excipients being designated the secondary excipient, tertiary excipient and so forth based on descending amounts by weight percent.

A pharmaceutical formulation of the present disclosure may further include a cyclic oligomer excipient, such as a cyclic oligosaccharide or cyclic oligosaccharide derivative excipient, a cyclic peptide oligomer or cyclic peptide oligomer derivative, or a cyclic polycarbonate oligomer or cyclic polycarbonate oligomer derivative, and any combinations thereof. An oligosaccharide excipient may have between 3 to 15 saccharide monomer units, such as glucose units and glucose derivative units, fructose units and fructose derivative units, galactose and galactose derivative units, and any combinations thereof. The saccharide monomer units may be derivatized with a functional group, for example a sulfobutylether, or a hydroxypropyl derivative, or a carboxymethyl derivative or by methylation. For example, the pharmaceutical formulation may include a cyclodextrin, such as a cyclodextrin containing 6, 7 or 8 monomer units, in particular an a cyclodextrin, such as CAVAMAX® W6 Pharma (Wacker Chemie AG, Germany), a β cyclodextrin, such as CAVAMAX® W7 Pharma (Wacker Chemie), or a γcyclodextrin, such as CAVAMAX® W8 Pharma (Wacker Chemie). Cyclodextrins contain dextrose units of (α-1,4)-linked α-D-glucopyranose that form acyclic structure having a lipophilic central cavity and a hydrophilic outer surface. Suitable cyclodextrins also include hydroxypropyl β cyclodextrin, such as KLEPTOSE® HBP (Roquette, France) and Na sulfo-butyl ether β cyclodextrin, such as DEXOLVE® 7 (Cyclolab, Ltd., Hungary).

Derivatization may facilitate the use of cyclic oligomer excipients in thermokinetic compounding.

Particularly when used in a thermokinetic compounding process, particle size of a cyclic oligomer excipient may facilitate compounding. Derivatization, pre-treatment, such by slugging or granulation, or both may increase or decrease particle size of a cyclic oligomer excipient to be within an optimal range. For example, the average particle size of a cyclic oligomer excipient may be increased by up to 500%, or up to 1,000%, by between 50% and 500%, or by between 50% and 1,000%. The average particle size of a cyclic oligomer excipient may be decreased by up to 50%, or up to 90%, or by between 5% and 50% or by between 5% and 90%.

The cyclic oligomer excipient may be used alone, or a pharmaceutical formulation of the present disclosure may include a combination of cyclic oligomer excipients.

A pharmaceutical formulation of the present disclosure may also include one or more additional excipients. These additional excipients may particularly include a polymer excipient or combination of polymer excipients. Suitable polymer excipients include may be water-soluble. Suitable polymer excipients may also be ionic or non-ionic.

Suitable polymer excipients include a cellulose-based polymer, a polyvinyl-based polymer, or an acrylate-based polymer. These polymers may have varying degrees of polymerization or functional groups.

Suitable cellulose-based polymers include an alkylcellulose, such as a methyl cellulose, a hydroxyalkylcellulose, or a hydroxyalkyl alkylcellulose. Suitable cellulose-based polymers more particularly include hydroxymethylcellulose, hydroxyethyl methylcellulose, hydroxyethylcellulose, hydroxypropylcellulose, hydroxybutylcellulose, hydroxypropylcellulose, hydroxypropylmethylcellulose, such as METHOCEL™ E3 and METHOCEL™ E5 (Dow Chemical, Michigan, US); ethylcellulose, such as ETHOCEL® (Dow Chemical), cellulose acetate butyrate, hydroxyethylcellulose, sodium carboxymethyl-cellulose, hydroxypropylmethylcellulose acetate succinate, such as AFFINISOL® HPMCAS 126 G (Dow Chemical), cellulose acetate, cellulose acetate phthalate, such as AQUATERIC™ (FMC, Pennsylvania, US), carboxymethylcellulose, such as sodium carboxymethycellulose, hydroxyethyl methyl cellulose, hydroxypropyl cellulose, hydroxyethyl cellulose, hydroxymethyl cellulose, and crystalline cellulose.

Suitable polyvinyl-based polymers include polyvinyl alcohol, such as polyvinyl alcohol 4-88, such as EMPROVE® (Millipore Sigma, Massachusetts, US) polyvinyl pyrrolidone, such as LUVITEK® (BASF, Germany) and KOLLIDON® 30 (BASF), polyvinylpyrrolidone-co-vinylacetate, poly(vinyl acetate)-co-poly(vinylpyrrolidone) copolymer, such as KOLLIDON® SR (BASF), poly(vinyl acetate) phthalate, such as COATERIC® (Berwind Pharmaceutical Services, Pennsylvania, US) or PHTHALAVIN® (Berwind Pharmaceutical Services), polyvinyl caprolactam-polyvinyl acetate-polyethylene glycol graft copolymer, such as SOLUPLUS® (BASF), polyvinyl caprolactam-polyvinyl acetate-polyethylene glycol graft copolymer, such as SOLUPLUS® (BASF), and hard polyvinylchloride.

Suitable acrylate-based polymers include acrylate and methacrylate copolymer, type A copolymer of ethylacrylate, methyl methacrylate and a methacrylic acid ester with quaternary ammonium groups in a ratio of 1:2:0.1, such as EUDRAGIT® RS PO (Evonik, Germany), poly(meth)acrylate with a carboxylic acid functional group, such as EUDRAGIT® 5100 (Evonik), dimethylaminoethyl methacrylate-methacrylic acid ester copolymer, ethylacrylate-methylmethacrylate copolymer, poly(methacrylate ethylacrylate) (1:1) copolymer, poly(methacrylate methylmethacrylate) (1:1) copolymer, poly(methacrylate methylmethacrylate) (1:2) copolymer, poly(methacrylic acid-co-ethyl acrylate) (1:1), such as EUDRAGIT® L-30-D (Evonik), poly(methacylic acid-co-ethyl acrylate) (1:1), such as EUDRAGIT® L100-55 (Evonik), poly(butyl methacylate-co-(2-dimethylaminoethyl) methacrylate-co-methyl methacrylate (1:2:1), such as EUDRAGIT® EPO (Evonik), methacrylic acid-ethacrylate copolymer, such as KOLLICOAT MAE 100-55 (BASF), polyacrylate, and polymethacrylate.

Certain polymer excipients are particularly well suited for use alone or in combinations as a secondary excipient with a cyclic oligomer primary excipient. The secondary polymer excipient may be water-soluble. The polymer secondary excipient may be ionic or non-ionic. Suitable secondary non-ionic polymer excipients include hydroxy propyl methyl cellulose, such as METHOCEL™ E15 (Dow Chemical, Michigan, US) or METHOCEL™ E50 (Dow Chemical), and polyvinylpyrrolidone, such as KOLLIDON® 90 (BASF, Germany). Suitable secondary ionic polymer excipients include hydroxy propyl methyl cellulose acetate succinate, such as AFFINISOL® HPMCAS 716 G (Dow Chemical), AFFINISOL® HPMCAS 912 G (Dow Chemical), and AFFINISOL® HPMCAS 126 G (Dow Chemical), polyvinyl acetate phthalate, such as PHTHALAVIN® (Berwind Pharmaceutical Services), and methacrylic acid based copolymer, such as methacrylic acid-ethacrylate copolymer, such as EUDRAGIT® L100-55 (Evonik, Germany).

One particularly well-suited secondary excipient includes hydroxy propyl methyl cellulose acetate succinate. The hydroxy propyl methyl cellulose acetate succinate may have 5-14%, more particularly 10-14%, and more particularly 12% acetate substitution. The hydroxy propyl methyl cellulose acetate succinate may have 4-18%, more particularly 4-8%, more particularly 6% succinate substitution.

A polymer excipient may include only one polymer, or a pharmaceutical formulation of the present disclosure may include a combination of polymer excipients.

Any excipient, including any cyclic oligomer excipient or any polymer excipient, in a pharmaceutical formulation of the present disclosure may also not contain substantial levels of impurities. For example, the excipient in a pharmaceutical formulation of the present disclosure may be have less than 1.0%, 0.75%, 0.5%, 0.1%, 0.05%, or 0.01% impurities by weight as compared to total weight of excipient and impurities, relative to a standard of known concentration in mg/mL. Impurities may include excipient degradation products, such as thermal degradation products.

A pharmaceutical formulation of the present disclosure may be in the form of an amorphous solid dispersion of the abiraterone and the excipient. The amorphous solid dispersion may contain less than 5% crystalline material, less than 1% crystalline material, or no crystalline material. The amorphous nature of the solid dispersion may be confirmed using X-ray diffraction (XRD), which may not exhibit strong peaks characteristic of a largely crystalline material.

A pharmaceutical formulation of the present disclosure may be formed by any suitable method for making amorphous solid dispersions, such as thermokinetic compounding, hot-melt extrusion, or spray drying. Thermokinetic compounding may be particularly useful for excipients that experience degradation in hot melt extrusion or that do not have a common organic solvent system with abiraterone as to facilitate spray drying.

A pharmaceutical formulation of the present disclosure containing amorphous abiraterone may dissolve more readily in the gastro-intestinal tract of a patient than a pharmaceutical formulation containing neat crystalline abiraterone, as evidenced by dissolution in at least one of 0.01 N HCl and biorelevant media, such as: Simulated Gastric Fluid (SGF), Fasted State Simulated Intestinal Fluid (FaSSIF), or Fed State Simulated Intestinal Fluid (FeSSIF).

Alternatively, the pharmaceutical formulation may be incorporated into a final dosage form that modifies or extends the release of abiraterone. This may include an extended release, delayed release, and/or pulsatile release profiles and the like. The pharmaceutical formulation may be incorporated into a tablet dosage from comprising a hydrophilic matrix that forms a swollen hydrogel in the gastric environment. This formation of hydrogel is intended to (1) retain the tablet in the stomach and (2) retard the release of abiraterone so as to provide a continuous release of the drug over a period of about 24 hours. More specifically, the dosage form, may be an extended release oral drug dosage form for releasing abiraterone into the stomach, duodenum and small intestine of a patient, and comprises: a single or a plurality of solid particles consisting of abiraterone or a pharmaceutically acceptable salt or prodrug or hydrate or solvate thereof dispersed within a polymer or a combination of polymers that (i) swells unrestrained dimensionally by imbibing water from gastric fluid to increase the size of the particles to promote gastric retention in the stomach of the patient in which the fasted/fed mode has been induced; (ii) gradually the abiraterone diffuses or the polymer erodes over a time period of hours, where the diffusion or erosion commences upon contact with the gastric fluid; herein the abiraterone ASD is vital for solubilization of abiraterone upon diffusion or erosion; and (iii) releases abiraterone to the stomach, duodenum and small intestine of the patient, as a result of the diffusion or polymeric erosion at a rate corresponding to the time period. Exemplary polymers include polyethylene oxides, alkyl substituted cellulose materials and combinations thereof, for example, high molecular weight polyethylene oxides and high molecular weight or viscosity hydroxypropylmethyl cellulose materials. A particularly well-suited polymer combination includes combination of polyethylene oxide POLYOX™ WSR 301 and hydroxypropyl methyl cellulose Methocel® E4M, used at ˜24% w/w and ˜18% w/w of the final tablet dosage form, respectively. This dosage from is intended to produce a pharmacokinetic profile with a reduced Cₘₐₓ-to-Cₘᵢₙ ratio such that human plasma concentrations remain within the therapeutic window for the duration of treatment. This abiraterone pharmacokinetic profile is expected to provide more efficacious cancer treatment with similar or reduced side effects.

The example above is only one example by which one can achieve a prolonged release of the solubility enhanced abiraterone ASD and thereby minimizing the Cₘₐₓ-to-Cₘᵢₙ ratio in a patient. Another example is a pulsatile release dosage form containing a component designed to release the solubility enhanced abiraterone ASD immediately in the stomach and one or more additional components designed to release a pulse of abiraterone at different regions in the intestinal tract. This can be accomplished by applying a pH-sensitive coating to one or more abiraterone ASD-containing components whereby the coating is designed to dissolve and release the active in different regions along the GI tract depending upon environmental pH. These functionally coated components may also contain an acidifying agent to decrease the microenvironmental pH to promote solubility and dissolution of abiraterone.

Furthermore, there are a myriad of controlled release technologies that could be applied to generate an extended abiraterone release profile when starting from the solubility enhanced abiraterone ASD compositions disclosed herein. It is important to note that the abiraterone ASD composition is enabling to this approach as applying conventional controlled drug release technologies to crystalline abiraterone or abiraterone acetate would fail to provide adequate drug release along the GI tract owing to the poor solubility of these forms of the compound.

In a pharmaceutical formulation of the present disclosure, the cyclic oligomer may be the only excipient. The pharmaceutical formulation may include 1% to 50% by weight amorphous abiraterone, particularly abiraterone, and between 50% and 99% by weight of one or more cyclic oligomer excipients. Alternatively, the pharmaceutical formulation may include at least 5%, at least 10%, or at least 20% by weight amorphous abiraterone, particularly abiraterone. Also alternatively, the pharmaceutical formulation may include at least 60% or at least 90% by weight of one or more cyclic oligomer excipients.

In another pharmaceutical formulation of the present disclosure, the cyclic oligomer may be the primary excipient. The pharmaceutical formulation may include 1% to 50% by weight amorphous abiraterone, particularly abiraterone, and between 50% and 99% by weight cyclic oligomer primary excipient. Alternatively, the pharmaceutical formulation may include at least 5%, at least 10%, or at least 20% by weight amorphous abiraterone, particularly abiraterone. Also alternatively, the pharmaceutical formulation may include at least 60% by weight cyclic oligomer excipient. The pharmaceutical formulation may further include at least 1% secondary excipient, particularly a polymer secondary excipient.

In another pharmaceutical formulation of the present disclosure, the cyclic oligomer may be the secondary excipient and the pharmaceutical formulation may further include a primary excipient, such as a polymer primary excipient. The pharmaceutical formulation may include 1% to 50% by weight amorphous abiraterone, particularly abiraterone, between 50% and 99% by weight primary excipient, and between 50% and 99% by weight cyclic oligomer secondary excipient. Alternatively, the pharmaceutical formulation may include at least 5%, at least 10%, or at least 20% by weight amorphous abiraterone, particularly abiraterone.

A pharmaceutical formulation of the present disclosure including abiraterone and a cyclic oligomer excipient, particularly a hydroxy propyl β cyclodextrin excipient in a molar ratio of abiraterone to cyclic oligomer excipient of 1:0.25 to 1:25, such as at least 1:2.

In a particular example, a pharmaceutical formulation of the present disclosure may be an amorphous dispersion of 1% to 50%, particularly at least 10% by weight abiraterone form, 80% by weight hydroxy propyl β cyclodextrin primary excipient, and 1% to 49%, particularly at least 10% by weight hydroxy propyl methyl cellulose acetate succinate secondary excipient.

A pharmaceutical formulation of the present disclosure may include an amount of amorphous abiraterone sufficient to achieve the same or greater therapeutic effect, bioavailability, Cₘᵢₙ, Cₘₐₓ or Tₘₐₓ as a greater amount of crystalline abiraterone or crystalline abiraterone acetate, such as ZYTIGA®, when consumed on an empty stomach. A pharmaceutical formulation as described herein may substantially improve the solubility of abiraterone, which may facilitate the improvement in therapeutic effect, bioavailability, Cₘᵢₙ, Cₘₐₓ or Tₘₐₓ.

“Therapeutic effect” may be measured by a decrease in measurable PSA level in a patient over a course of treatment, such as a one-month course of treatment. Other scientifically accepted measures of therapeutic effect, such as those used in the course of obtaining regulatory approval, particularly FDA approval, may also be used to determine “therapeutic effect.”

“Bioavailability” is measured herein as the area under the drug plasma concentration versus time curve (AUC) from an administered unit dosage form. Absolute bioavailability is the bioavailability of an oral composition compared to an intravenous reference assumed to deliver 100% of the active into systemic circulation. The insolubility of abiraterone precludes intravenous delivery; therefore, the absolute bioavailability of abiraterone cannot be known. The absolute bioavailability of ZYTIGA® when administered as approved on an empty stomach must be less than 10% because its AUC increases 10-fold when administered with a high-fat meal. The increase in bioavailability of ZYTIGA® when administered with a high-fat meal is assumed to be the result of improved solubility of abiraterone acetate in the fed state. In order to facilitate comparisons, bioavailability in the present disclosure may be measured on an empty stomach, such as at least two hours after the last ingestion of food and at least one hour before the next ingestion of food.

For example, the relative bioavailability of abiraterone in a pharmaceutical formulation of the present disclosure as compared to ZYTIGA® or a comparable crystalline abiraterone acetate may be at least 500% greater or even at least 1,000% greater.

In particular, a pharmaceutical formulation of the present disclosure may include an amount of amorphous abiraterone sufficient to achieve the same therapeutic effect or the same bioavailability in a patient as 1000 mg of crystalline abiraterone acetate, such as ZYTIGA®, when consumed on an empty stomach, once daily. Such a pharmaceutical formulation may also include a glucocorticoid replacement API, such as 5 mg of glucocorticoid replacement API.

Alternatively, a pharmaceutical formulation of the present disclosure may include an amount of amorphous abiraterone sufficient to achieve the same therapeutic effect or the same bioavailability in a patient as 500 mg of crystalline abiraterone acetate, such as ZYTIGA®, when consumed on an empty stomach, twice daily. Such a pharmaceutical formulation may also include a glucocorticoid replacement API, such as 5 mg of glucocorticoid replacement API.

A pharmaceutical formulation of the present disclosure may be for oral administration and may be further processed, with or without further compounding, to facilitate oral administration.

A pharmaceutical formulation of the present disclosure may be further processed into a solid dosage form suitable for oral administration, such as a tablet or capsule.

In order to further increase therapeutic effect, bioavailability, Cₘᵢₙ, or Cₘₐₓ of the abiraterone, a pharmaceutical formulation of the present disclosure may be combined with an additional amount of the primary excipient, secondary (or tertiary, etc.) excipient, such as hydroxy propyl methyl cellulose acetate secondary excipient, or another suitable concentration enhancing polymer not part of the pharmaceutical formulation to produce the solid dosage form.

Concentration enhancing polymers suitable for use in the solid dosage form may include compositions that do not interact with abiraterone in an adverse manner. The concentration enhancing polymer may be neutral or ionizable. The concentration enhancing polymer may have an aqueous solubility of at least 0.1 mg/mL over at least a portion of or all of pH range 1-8; particularly at least a portion of or all of pH range 1-7 or at least a portion of or all of pH range 7-8. When the solid dosage form is dissolved in 0.01 N HCl and biorelevant media, such as: Simulated Gastric Fluid (SGF), Fasted State Simulated Intestinal Fluid (FaSSIF), or Fed State Simulated Intestinal Fluid (FeSSIF), the concentration-enhancing polymer may increase the maximum abiraterone concentration dissolved in the biorelevant media by a factor of at least 1.25, at least 2, or at least 3 as compared to an identical solid dosage form lacking the concentration enhancing polymer. A similar increase in maximum abiraterone concentration in biorelevant media may be observed when additional primary or secondary (or tertiary, etc.) excipients not present in the pharmaceutical formulation are added to the dosage form.

### B. Methods of Formulating a Pharmaceutical Formulation

A pharmaceutical formulation of the present disclosure may be prepared using thermokinetic compounding, which is a method of compounding components until they are melt-blended. Thermokinetic compounding may be particularly useful for compounding heat-sensitive or thermolabile components. Thermokinetic compounding may provide brief processing times, low processing temperatures, high shear rates, and the ability to compound thermally incompatible materials.

Thermokinetic compounding may be carried out in a thermokinetic chamber using one or multiple speeds during a single, compounding operation on a batch of components to form a pharmaceutical formulation of the present disclosure.

A thermokinetic chamber includes a chamber having an inside surface and a shaft extending into or through the chamber. Extensions extend from the shaft into the chamber and may extend to near the inside surface of the chamber. The extensions are often rectangular in cross-section, such as in the shape of blades, and have facial portions. During thermokinetic compounding, the shaft is rotated causing the components being compounded, such as particles of the components being compounded, to impinge upon the inside surface of the chamber and upon facial portions of the extensions. The shear of this impingement causes comminution, frictional heating, or both of the components and translates the rotational shaft energy into heating energy. Any heating energy generated during thermokinetic compounding is evolved from the mechanical energy input. Thermokinetic compounding is carried out without an external heat source. The thermokinetic chamber and components to be compounded are not pre-heated prior to commencement of thermokinetic compounding.

The thermokinetic chamber may include a temperature sensor to measure the temperature of the components or otherwise within the thermokinetic chamber.

During thermokinetic compounding, the average temperature of the thermokinetic chamber may increase to a pre-defined final temperature over the duration of the thermokinetic compounding to achieve thermokinetic compounding of the abiraterone and the excipient, and any other components of a pharmaceutical formulation of the present disclosure, such as an additional API, for example a glucocorticoid replacement API, an additional excipient, or both. The pre-defined final temperature may be such that degradation of the abiraterone, excipient, or other components is avoided or minimized. Similarly, the one or multiple speeds of use during thermokinetic compounding may be such that thermal degradation of the abiraterone, excipient, or other components is avoided or minimized. As a result, the abiraterone, excipient, or other components of the solid amorphous dispersion may lack substantial impurities.

The average maximum temperature in the thermokinetic chamber during thermokinetic compounding may be less than the glass transition temperature, melting point, or molten transition point, of abiraterone or any other APIs present, one or all excipients, or one or all other components of the amorphous solid dispersion, or any combinations or sub-combinations of components.

Pressure, duration of thermokinetic compounding, and other environmental conditions such as pH, moisture, buffers, ionic strength of the components being mixed, and exposure to gasses, such as oxygen, may also be such that degradation of abiraterone or any other APIs present, one or all excipients, or one or all other components is avoided or minimized.

Thermokinetic compounding may be performed in batches or in a semi-continuous fashion, depending on the product volume. When performed in a batch, semi-continuous, or continuous manufacturing process, each thermokinetic compounding step may occur for less than 5, 10, 15, 20, 25, 30, 35, 40, 45, 50, 55, 60, 70, 100, 120, 240, or 300 seconds.

Variations of thermokinetic compounding may be used depending on the amorphous solid dispersion and its components. For example, the thermokinetic chamber may be operated at a first speed to achieve a first process parameter, then operated at a second speed in the same thermokinetic compounding process to achieve a final process parameter. In other examples, the thermokinetic chamber may be operated at more than two speeds, or at only two speeds, but in more than two time internals, such as at a first speed, then at a second speed, then again at the first speed.

The abiraterone component may be in a crystalline or semi-crystalline form prior to thermokinetic compounding.

In another variation, abiraterone or other API particle size is reduced prior to thermokinetic compounding. This may be accomplished by milling, for example dry milling the crystalline form of the abiraterone or other API to a small particle size prior to thermokinetic compounding, wet milling the crystalline form of the abiraterone or other API with a pharmaceutically acceptable solvent to reduce the particle size prior to thermokinetic compounding, or melt milling the crystalline form of the abiraterone or other API with at least one excipient having limited miscibility with the crystalline form of the abiraterone or other API to reduce the particle size prior to thermokinetic compounding.

Another variation includes milling the crystalline form of the abiraterone or other API in the presence of an excipient to create an ordered mixture where the abiraterone or other API particles adhere to the surface of excipient particles, excipient particles adhere to the surface of API particles, or both.

The thermokinetically compounded amorphous solid dispersion may exhibit substantially complete amorphicity.

A pharmaceutical formulation of the present disclosure may be prepared using hot melt extrusion, whereby an excipient blend is heated to a molten state and subsequently forced through an orifice where the extruded product is formed into its final shape in which it is solidified upon cooling. The blend is conveyed through various heating zones typically by a screw mechanism. The screw or screws are rotated by a variable speed motor inside a cylindrical barrel where only a small gap exists between the outside diameter of the screw and the inside diameter of the barrel. In this conformation, high shear is created at the barrel wall and between the screw fights by which the various components of the powder blend are well mixed and deaggregated.

The hot-melt extrusion equipment is typically a single or twin-screw apparatus but can be composed of more than two screw elements. A typical hot-melt extrusion apparatus contains a mixing/conveying zone, a heating/melting zone, and a pumping zone in succession up to the orifice. In the mixing/conveying zone, the powder blends are mixed and aggregates are reduced to primary particles by the shear force between the screw elements and the barrel. In the heating/melting zone, the temperature is at or above the melting point or glass transition temperature of the thermal binder or binders in the blend such that the conveying solids become molten as they pass through the zone. A thermal binder in this context describes an inert excipient, typically a polymer, that is solid at ambient temperature, but becomes molten or semi-liquid when exposed to elevated heat or pressure. The thermal binder acts as the matrix in which the abiraterone and other APIs are dispersed, or the adhesive with which they are bound such that a continuous composite is formed at the outlet orifice. Once in a molten state, the homogenized blend is pumped to the orifice through another heating zone that maintains the molten state of the blend. At the orifice, the molten blend may be formed into strands, cylinders or films. The extrudate that exits is then solidified typically by an air-cooling process. Once solidified, the extrudate may then be further processed to form pellets, spheres, fine powder, tablets, and the like.

A pharmaceutical formulation as disclosed herein resulting from hot melt extrusion may have a uniform shape and density and may not exhibit substantially changed solubility or functionality of any excipient. The abiraterone, excipient, or other components of the pharmaceutical formulation may lack substantial impurities.

A pharmaceutical formulation of the present disclosure may be prepared using spray drying. In the spray-drying process, components, including abiraterone, an excipient and any other APIs or excipients are dissolved in a common solvent which dissolves the components to produce a mixture. After the components have been dissolved, the solvent is rapidly removed from the mixture by evaporation in the spray-drying apparatus, resulting in the formation of a solid amorphous dispersion of the components. Rapid solvent removal is accomplished by either (1) maintaining the pressure in the spray-drying apparatus at a partial vacuum (e.g., 0.01 to 0.50 atm); (2) mixing the mixture with a warm drying gas; or (3) both (1) and (2). In addition, a portion or all of the heat required for solvent evaporation may be provided by heating the mixture.

Solvents suitable for spray-drying can be any organic compound in which the abiraterone and primary excipient and any additional APIs or excipients are mutually soluble. The solvent may also have a boiling point of 150° C. or less. In addition, the solvent should have relatively low toxicity and be removed from the dispersion to a level that is acceptable according to The International Committee on Harmonization (ICH) guidelines, which are incorporated by reference herein. A further processing step, such as tray-drying subsequent to the spray-drying process, may be used to remove solvent to a sufficiently low level.

Suitable solvents include alcohols such as methanol, ethanol, n-propanol, iso-propanol, and butanol; ketones such as acetone, methyl ethyl ketone and methyl iso-butyl ketone; esters such as ethyl acetate and propylacetate; and various other solvents such as acetonitrile, methylene chloride, toluene, and 1,1,1-trichloroethane. Lower volatility solvents such as dimethylacetamide or dimethylsulfoxide may also be used. Mixtures of solvents may also be used, as may mixtures with water as long as the abiraterone, excipient, and any other APIs or excipients in the pharmaceutical formulation are sufficiently soluble to allow spray-drying.

The abiraterone, excipient, or other components of a pharmaceutical formulation as disclosed herein resulting spray-drying may lack substantial impurities.

Following formulation of a pharmaceutical formulation as disclosed herein, an amount appropriate to provide a given unit dosage form may be further processed, for example to result in an orally administrable form. This further processing may include combining the pharmaceutical formulation as an internal phase with an external phase, if needed, along with tableting by a tableting press or encapsulation in a capsule. The external phase may include an additional amount of an excipient or a concentration enhancing polymer to further improve, for example, the therapeutic effect, bioavailability, Cₘᵢₙ, or Cₘₐₓ.

In some examples, the pharmaceutical formulation may be tableted, then coated with a composition containing another API, such as a glucocorticoid replacement API.

### C. Methods of Administering a Pharmaceutical Formulation

The FDA-approved form of crystalline abiraterone acetate, ZYTIGA®, is administered on an empty stomach to prostate cancer patients at a total dose of 1,000 mg once daily, as multiple unit dosage form tablets. The bioavailability of ZYTIGA® at these conditions is estimated to be <10%.

Recent studies have indicated that the low oral bioavailability of ZYTIGA® may be responsible for poor clinical outcomes in a significant portion of the patient population. This has been demonstrated by correlating steady-state minimum serum concentration (Cₘᵢₙ) to reductions in PSA levels. In the treatment of mCRPC with ZYTIGA®, reductions in PSA are predictive of improved clinical outcomes. Early response, such as a PSA decline >30% from baseline by 4 weeks, is associated with longer overall survival. Robust response, such as a PSA decline >50% from baseline at 12 weeks is associated with longer overall survival. However, a significant proportion of ZYTIGA® patients do not achieve robust PSA reductions.

In a Phase 3 study in chemotherapy naïve patients (COU-AA-302), 38% of subjects (208 of 542) did not achieve PSA decline >50% according to Prostate Cancer Clinical Trials Working Group (PCWG2) criteria. In a Phase 3 study in prior docetaxel treated patients (COU-AA-301), 61% of patients (632 of 790) did not achieve a PSA decline >50% according to PCWG2 criteria. In a Phase 3 study of enzalutamide in prior docetaxel treated patients (AFFIRM), patients progressing on enzalutamide were subsequently offered salvage therapy with ZYTIGA®: only 8% (3 of 37) of the patients achieved PSA decline >50%.

Better PSA response with ZYTIGA® treatment is associated with patients who have higher Cₘᵢₙ of abiraterone. In a tumor-inhibition model built upon pooled data from the COU-AA-301 and COU-AA-302 Phase 3 studies, patients with higher Cₘᵢₙ of abiraterone had longer time until PSA progression (PSA Doubling Time) which was predictive of longer overall survival. In a FDA regulatory review analysis of COU-AA-301 trial data, subjects in the group having higher Gnu, of abiraterone showed a trend towards longer overall survival. These results suggest that increasing Cₘᵢₙ levels by increasing overall abiraterone exposure may lead to improved clinical outcomes with abiraterone.

When administered with a high fat meal, oral exposure increases substantially, with maximum serum concentration (Cₘₐₓ) and area under the plasma drug concentration-time curve (AUC) being 17 and 10-fold higher, respectively. Recent studies have indicated that this substantial food effect results from increased solubility of abiraterone acetate, such as ZYTIGA® and abiraterone in intestinal fluids of the fed state. Owing to the magnitude of this food effect and variation in meal content, ZYTIGA® must be taken on an empty stomach.

The abiraterone acetate prodrug form of abiraterone, such as ZYTIGA®, was developed to improve the solubility and bioavailability of abiraterone. However, the effectiveness of the prodrug toward improving bioavailability is limited, as evidenced by the food effect and pharmacokinetic variability cited in the label. Further, exposure was not significantly increased when the Zytiga dose was doubled from 1,000 to 2,000 mg (8% increase in the mean AUC). The results of this study imply that Zytiga is dosed near the absorption limit. A pharmaceutical formulation of the present disclosure may contain amorphous abiraterone, such as the active form of abiraterone, which may exhibit improved therapeutic effect, bioavailability, Cₘᵢₙ, or Cₘₐₓ as compared to an equivalent amount of crystalline abiraterone or an equivalent amount of crystalline abiraterone acetate.

A pharmaceutical formulation of the present disclosure may be administered in a dosage form, such as a unit dosage form containing an amount of abiraterone sufficient and at a frequency sufficient to achieve a greater therapeutic effect, the same or greater bioavailability, the same or greater Cₘᵢₙ, or the same or greater Cₘₐₓ as an equivalent amount of crystalline abiraterone acetate, such as ZYTIGA®, administered at the same frequency.

A pharmaceutical formulation of the present disclosure may be administered in a dosage form, such as a unit dosage form containing an amount of abiraterone sufficient and at a frequency sufficient to achieve the same or greater therapeutic effect, bioavailability, Cₘᵢₙ, or Cₘₐₓ as crystalline abiraterone acetate, such as ZYTIGA®, administered at 1000 mg once daily on an empty stomach.

At 1,000 mg daily, after multiple days of dosing with ZYTIGA®, patients with mCRPC showed inter-subject variability of 79% for Cₘₐₓ and 64% for AUC₀₋₂₄ₕ. Administration of a pharmaceutical formulation of the present disclosure, for example in a unit dosage form, may result in at least a 10% decrease, at least a 20% decrease, at least a 30% decrease, at least a 40% decrease, at least a 50% decrease, at least a 60% decrease, at least a 70% decrease, at least an 80% decrease, or at least a 90% decrease in variability among patients with a response within two standard deviations of the average response in therapeutic effect, bioavailability, Cₘᵢₙ, or Cₘₐₓ as compared to an administration of an equivalent amount of crystalline abiraterone or crystalline abiraterone acetate, such as ZYTIGA®.

Administration of ZYTIGA® with a high-fat meal increased the geometric mean of Cₘₐₓ by 17-fold and AUC_{0-∞} by 10-fold. Administration of a pharmaceutical formulation of the present disclosure, for example in a unit dosage form, may result in at least a 10% decrease, at least a 20% decrease, at least a 30% decrease, at least a 40% decrease, at least a 50% decrease, at least a 60% decrease, at least a 70% decrease, at least an 80% decrease, or at least a 90% decrease in fasting-state vs. high fat meal variability in therapeutic effect, bioavailability, Cₘᵢₙ, or Cₘₐₓ as compared to an administration of an equivalent amount of crystalline abiraterone or crystalline abiraterone acetate, such as ZYTIGA®.

The above and other improvements may be due, at least in part, to improved solubility of abiraterone when present in a pharmaceutical formulation as of the present disclosure, as compared to the solubility of crystalline abiraterone or crystalline abiraterone acetate, such as ZYTIGA®, in other formulations.

Abiraterone is typically co-administered with a glucocorticoid replacement API, such as prednisone, methylprednisone, or prednisolone. For example, abiraterone acetate, such as ZYTIGA®, is typically co-administered with twice daily doses of 5 mg of prednisone, methylprednisone, or prednisolone. Methyprednisolone and dexamethasone may also be suitable glucocorticoid replacement APIs and may be administered in similar doses or doses calculated to achieve a similar glucocorticoid replacement effect as prednisone, methylprednisone, or prednisolone, in particular twice-daily administration of 5 mg of prednisone, methylprednisone, or prednisolone.

Abiraterone is the active metabolite of abiraterone acetate and is expected to have the same or similar biological effects as abiraterone acetate, such as ZYTIGA®, and thus may be administered with a glucocorticoid replacement API on a similar dosing schedule.

A pharmaceutical formulation of the present disclosure may further include a glucocorticoid replacement API, such as prednisone, methylprednisone, prednisolone, methylprednisolone, or dexamethasone, or other alkylated forms, along with the abiraterone and excipient or excipients.

A pharmaceutical formulation of the present disclosure may include 1000 mg of amorphous abiraterone or an amount of amorphous abiraterone sufficient to achieve the same or greater therapeutic effect, bioavailability, Cₘᵢₙ, or Cₘₐₓ in a patient as 1000 mg of crystalline abiraterone or crystalline abiraterone acetate, such as ZYTIGA®, when consumed on an empty stomach. Such a formulation may be designed for once-daily administration. Administration may be combined with co-administration of the glucocorticoid replacement API, such as prednisone, methylprednisone, prednisolone, methylprednisolone, or dexamethasone, for example twice daily.

A pharmaceutical formulation of the present disclosure may include 1000 mg of amorphous abiraterone, or an amount of amorphous abiraterone sufficient to achieve the same or greater therapeutic effect, bioavailability, Cₘᵢₙ, or Cₘₐₓ in a patient as 1000 mg of crystalline abiraterone or crystalline abiraterone acetate, such as ZYTIGA®, when consumed on an empty stomach, along with a glucocorticoid replacement API, such as prednisone, methylprednisone, prednisolone, methylprednisolone, or dexamethasone, for example in a 5 mg amount. Such a formulation may be designed for once-daily administration, combined with co-administration of the glucocorticoid replacement API, such as prednisone, methylprednisone, prednisolone, methylprednisolone, or dexamethasone, for example in a 5 mg amount, once additionally daily.

A pharmaceutical formulation of the present disclosure may include 500 mg of amorphous abiraterone, or an amount of amorphous abiraterone sufficient to achieve the same or greater therapeutic effect, bioavailability, Cₘᵢₙ, or Cₘₐₓ in a patient as 500 mg of crystalline abiraterone or crystalline abiraterone acetate, such as ZYTIGA®, when consumed on an empty stomach. Such a formulation may be designed for twice-daily administration or for administration of two unit dosage forms once daily. Administration may be combined with co-administration of the glucocorticoid replacement API, such as prednisone, methylprednisone, prednisolone, methylprednisolone, or dexamethasone, for example in 5 mg amounts, for example twice daily.

A pharmaceutical formulation of the present disclosure may include 500 mg of amorphous abiraterone, or an amount of amorphous abiraterone sufficient to achieve the same or greater therapeutic effect, bioavailability, Cₘᵢₙ, or Cₘₐₓ in a patient as 500 mg of crystalline abiraterone or crystalline abiraterone acetate, such as ZYTIGA®, when consumed on an empty stomach, along with a glucocorticoid replacement API, such as prednisone, methylprednisone, prednisolone, methylprednisolone, or dexamethasone, for example in 2.5 mg amounts. Such a formulation may be designed for twice-daily administration. Such a formulation may be combined with co-administration of the glucocorticoid replacement API, such as prednisone, methylprednisone, prednisolone, methylprednisolone, or dexamethasone, for example in a 5 mg amount, once additionally daily.

A pharmaceutical formulation of the present disclosure may include 250 mg of amorphous abiraterone, or an amount of amorphous abiraterone sufficient to achieve the same or greater therapeutic effect, bioavailability, Cₘᵢₙ, or Cₘₐₓ in a patient as 250 mg of crystalline abiraterone or crystalline abiraterone acetate, such as ZYTIGA®, when consumed on an empty stomach. Such a formulation may be designed for administration of two-unit dosage forms twice daily or for administration of four unit dosage forms once daily. Administration may be combined with co-administration of the glucocorticoid replacement API, such as prednisone, methylprednisone, prednisolone, methylprednisolone, or dexamethasone, for example in 5 mg amounts, for example twice daily.

A pharmaceutical formulation of the present disclosure may include 250 mg, 200 mg, 150 mg, 100 mg, 70 mg, 50 mg. 25 mg or 10 mg of amorphous abiraterone, including ranges of 10 mg to 70 mg, 25 mg to 70 mg, or 50 mg to 70 mg, or an amount of amorphous abiraterone sufficient to achieve the same or greater therapeutic effect, bioavailability, Cₘᵢₙ, or Cₘₐₓ in a patient as 1000, 500 mg, 250 mg, 200 mg, 150 mg, 100 mg, 50 mg or 25 mg of crystalline abiraterone or crystalline abiraterone acetate, such as ZYTIGA®, when consumed on an empty stomach, along with a glucocorticoid replacement API, such as prednisone, methylprednisone, prednisolone, methylprednisolone, or dexamethasone, for example in 1.25 mg amounts. Such a formulation may be designed for twice-daily administration. Such a formulation may be designed for twice-daily administration. Such a formulation may be combined with co-administration of the glucocorticoid replacement API, such as prednisone, methylprednisone, prednisolone, methylprednisolone, or dexamethasone, for example in a 5 mg amount, once additionally daily.

Variations of the above example formulations and dosing regimens are possible. For example, amounts of abiraterone, glucocorticoid replacement API, or both, in a pharmaceutical formulation may be varied based upon the intended administration schedule.

Although prednisone, methylprednisone, prednisolone, methylprednisolone, or dexamethasone and alkylated forms thereof are recited as specific glucocorticoid replacement APIs, other glucocorticoid replacement APIs may also be used. Combinations of glucocorticoid APIs may be used, whether in the pharmaceutical formulation of co-administered.

In general, a pharmaceutical formulation of the present disclosure may be used to administer any amount of abiraterone to a patient on any schedule. In addition, any pharmaceutical formulation of the present disclosure may be co-administered with any other API, whether or not in the pharmaceutical formulation, that also treats prostate cancer, a side-effect of abiraterone, or a side-effect of prostate cancer. Co-administered APIs may include a glucocorticoid replacement API or another API to treat prostate cancer, such as APIs used in androgen-deprivation therapy, non-steroidal androgen receptor inhibitors, taxanes, gonadotrophin-releasing hormone antagonists, gonadotropin-releasing hormone analogs, androgen receptor antagonists, non-steroidal anti-androgens, analogs of luteinizing hormone-releasing hormone, anthracenedione antibiotics, and radiopharmaceuticals, and any combinations thereof, particularly bicalutamide, such as CASODEX® (AstraZenica, North Carolina, US) cabazitaxel, such as JEVTANA® (Sanofi-Aventis, France), degarelix, docetaxel, such as TAXOTERE® (Sanofi-Aventis), enzalutamide, such as XTANDI® (Astellas Pharma, Japan), flutamide, goserelin acetate, such as ZOLADEX® (TerSera Therapeutics, Iowa, US), leuprolide acetate, such as LUPRON® (Abbvie, Ill., US), LUPRON® DEPOT (Abbive), LUPRON® DEPOT-PED (Abbive), and VIADUR® (ALZA Corporation, California, US), mitoxantrone hydrochloride, nilutamide, such as NILANDRON® (Concordia Pharmaceuticals, Barbados), and radium 223 dichloride, such a XOFIGO® (Bayer Healthcare Pharmaceuticals, New Jersey, US), and any combinations thereof.

Amorphous abiraterone in a pharmaceutical formulation of the present disclosure, may be administered using fewer or smaller tablets or capsules than is possible with formulations crystalline abiraterone acetate, such as ZYTIGA®, which may increase patient compliance and decrease patient discomfort.

A pharmaceutical formulation of the present disclosure may be particularly useful when the patient has experienced a sub-optional response to formulations containing crystalline abiraterone acetate, such as ZYTIGA®.

A pharmaceutical formulation of the present disclosure may be administered to a patient with prostate cancer, such as a patient with castration-resistant prostate cancer, metastatic castration-resistant prostate cancer, metastatic prostate cancer, locally advanced prostate cancer, relapsed prostate cancer, or other high-risk prostate cancer.

A pharmaceutical formulation of the present disclosure may be administered to a patient with prostate cancer who has previously received treatment with chemotherapy, such as docetaxel.

A pharmaceutical formulation of the present disclosure may be administered to a patient with prostate cancer who has previously received treatment with enzalutamide.

A pharmaceutical formulation of the present disclosure may be administered to a patient in combination with androgen-deprivation therapy.

A pharmaceutical formulation of the present disclosure may be administered to a patient with breast cancer.

A pharmaceutical formulation of the present disclosure may be administered to a patient with breast cancer who has previously received treatment with chemotherapy, such as docetaxel.

A pharmaceutical formulation of the present disclosure may be administered to a patient with breast cancer who has previously received treatment with enzalutamide.

A pharmaceutical formulation of the present disclosure may be administered to a patient in combination with androgen-deprivation therapy.

A pharmaceutical formulation of the present disclosure may be administered to a patient with salivary gland cancer.

A pharmaceutical formulation of the present disclosure may be administered to a patient with salivary gland cancer who has previously received treatment with chemotherapy, such as docetaxel.

A pharmaceutical formulation of the present disclosure may be administered to a patient with salivary gland cancer who has previously received treatment with enzalutamide.

A pharmaceutical formulation of the present disclosure may be administered to a patient in combination with androgen-deprivation therapy.

A pharmaceutical formulation of the present disclosure may be administered to a patient with a cancer known to respond to androgen deprivation therapy.

A pharmaceutical formulation of the present disclosure may be administered to a patient with a cancer known to respond to androgen deprivation therapy who has previously received treatment with chemotherapy, such as docetaxel.

A pharmaceutical formulation of the present disclosure may be administered to a patient with a cancer known to respond to androgen deprivation therapy who has previously received treatment with enzalutamide.

A pharmaceutical formulation of the present disclosure may be administered to a patient in combination with additional androgen-deprivation therapy.

Any of the pharmaceutical formulations maybe administered in one or more tablets.

### D. Examples

The present examples are provided for illustrative purposes only. They are not intended to and should not be interpreted to encompass the full breadth of the disclosure.

Various compositions and instruments are identified by trade name in this application. All such trade names refer to the relevant composition or instrument as it existed as of the earliest filing date of this application, or the last date a product was sold commercially under such trade name, whichever is later. One of ordinary skill in the art will appreciate that variant compositions and instruments sold under the trade name at different times will typically also be suitable for the same uses.

### Example 1: Solid Dispersions of Abiraterone with Various Polymer Excipients

Solid dispersions, some of which were amorphous solid dispersions and some of which were not (at the investigated processing conditions), were prepared via thermokinetic compounding using a lab-scale thermokinetic compounder (DisperSol Technologies LLC, Austin, Tex.). 10% by weight neat crystalline abiraterone was physically mixed with 90% by weight polymer excipient by hand-blending for two minutes in a polyethylene bag. Polymer excipients varied as indicated in Table 1. The binary mixture was then thermokinetically compounded with an ejection temperature of between 120° C.-230° C. During thermokinetic compounding, the material was subjected to a range of shear stresses controlled by a computer algorithm, with defined rotational speeds. When the ejection temperature was reached, the resulting thermokinetically processed solid dispersion (KSD) was automatically discharged into a catch tray and immediately quenched between two stainless steel plates. Thermokinetic compounding outcomes are further described in Table 1.

TABLE 1
Abiraterone-polymer excipient solid dispersions and
thermokinetic compounding outcomes

| | Composition | | |
|---|---|---|---|
| Ex. | API | | |
| No. | (10% Wt) | Polymer (90% Wt) | Outcome |

Cellulose based

| | | | |
|---|---|---|---|
| 1.1 | Abiraterone | Hydroxy Propyl Methyl | Fully Processed |
| | | Cellulose- | |
| | | METHOCEL ™ E3 | |
| 1.2 | Abiraterone | Hydroxy Propyl Methyl | Fully Processed |
| | | Cellulose- | |
| | | METHOCEL ™ E5 | |
| 1.3 | Abiraterone | Hydroxy Propyl Methyl | Fully Processed |
| | | Cellulose Acetate Succinate- | |
| | | AFFINISOL ® HPMCAS 126 G | |

Polyvinyl based

| | | | |
|---|---|---|---|
| 1.4 | Abiraterone | Polyvinyl Pyrrolidone- | Fully Processed |
| | | KOLLIDON ® 30 | |
| 1.5 | Abiraterone | Polyvinyl Acetate Phthalate- | Fully Processed |
| | | PHTHALAVIN ® | |
| 1.6 | Abiraterone | Polyvinyl Alcohol 4-88- | Fully Processed |
| | | EMPROVE ® | |

Acrylate based

| | | | |
|---|---|---|---|
| 1.7 | Abiraterone | Methacrylic Acid-Ethylacrylate | Fully Processed |
| | | copolymer- | |
| | | KOLLICOAT MAE ® 100-55 | |

The KSDs were further milled to a powder using a lab-scale rotor mill (IKA mill, IKA Works GmbH & Co. KG, Staufen, Germany) equipped with 20 ml grinding chamber and operated between 10000 rpm to 24000 rpm for a period of 60 seconds at a time. The milled KSDs were sieved and the particle size fraction of ≤250 μm was used for further analysis.

The neat crystalline abiraterone and KSDs were analyzed for their crystalline character by XRD using a Rigaku MiniFlex 600 benchtop X-ray diffractometer (Rigaku, Inc., Tokyo, Japan). Samples were loaded into an aluminum pan, leveled with a glass slide and analyzed in the 2-theta range between 2.5°-40.0° while being spun. The step size was 0.02°, and the scanning rate was set to 5.0°/min. The following additional instrument settings were used: Slit condition: variable+fixed slit system; soller (inc.): 5.0°; IHS: 10.0 mm; DS: 0.625°; SS: 8.0 mm; soller (rec.): 5.0°; RS: 13.0 mm (Open); monochromatization: kb filter (x2); voltage: 40 kV; current: 15 mA.

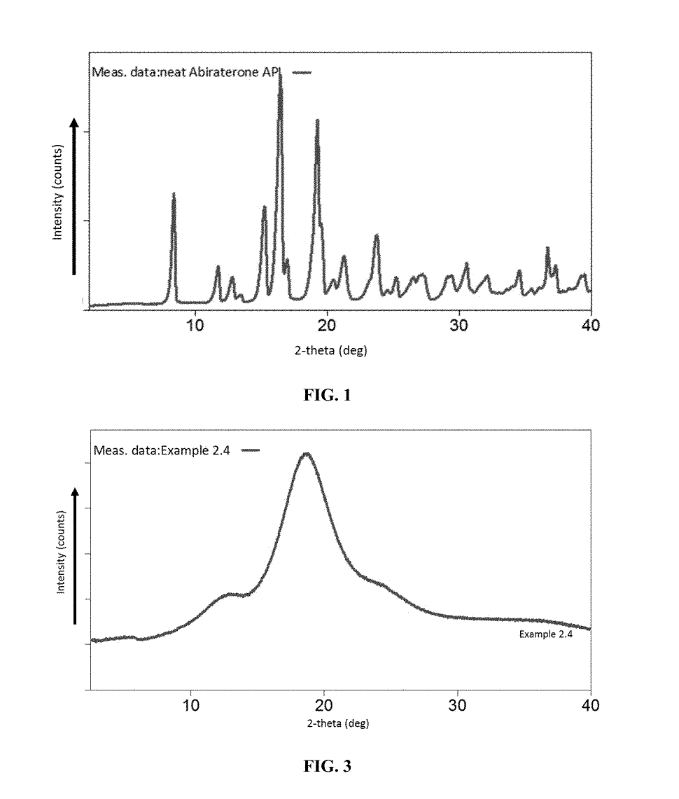

XRD diffractograms for neat crystalline abiraterone and the various KSDs are presented in FIGS. 1 and 2.

Neat crystalline abiraterone was processable via thermokinetic compounding with all three general types of polymer excipients tested. Comparing the X-ray diffractogram of neat crystalline abiraterone (FIG. 1), with X-ray diffractograms of the KSDs (FIG. 2), shows that in the cellulose-based polymer excipient group, hydroxy propyl methyl cellulose with varying viscosities yielded amorphous solid dispersions, whereas hydroxy propyl methyl cellulose acetate succinate yielded a KSD with substantially reduced crystallinity. Among the polyvinyl-based polymer excipient group, polyvinyl pyrrolidone and polyvinyl acetate phthalate produced amorphous solid dispersions, while polyvinyl alcohol 4-88 yielded a KSD with substantially reduced crystallinity. The methacrylic acid-ethylacrylate copolymer-based polymer excipient produced an amorphous solid dispersion.

### Example 2: Solid Dispersions of Abiraterone with Various Cyclic Oligomer Excipients

Various KSDs of abiraterone and cyclic oligomer excipients were prepared as in Example 1. Cyclic oligomer excipients and thermokinetic compounding outcomes are described in Table 2.

TABLE 2
Abiraterone-cyclic oligomer excipient solid dispersions and thermokinetic
compounding outcomes

| Ex. | Composition | | |
|---|---|---|---|
| No. | API (10% Wt) | Cyclic Oligomer (90% Wt) | Outcome |

Native cyclic oligomer

| | | | |
|---|---|---|---|
| 2.1 | Abiraterone | α-Cyclodextrin- | Under processed |
| | | CAVAMAX ® W6 Pharma | |
| 2.2 | Abiraterone | β-Cyclodextrin- | Under processed |
| | | CAVAMAX ® W7 Pharma | |
| 2.3 | Abiraterone | γ-Cyclodextrin- | Under processed |
| | | CAVAMAX ® W8 Pharma | |

Modified cyclic oligomer

| | | | |
|---|---|---|---|
| 2.4 | Abiraterone | Hydroxy Propyl β | Fully processed |
| | | Cyclodextrin- | |
| | | KLEPTOSE ® HPB | |
| 2.5 | Abiraterone | Sulfo butyl β Cyclodextrin | Under processed |
| | | Sodium Salt- | |
| | | DEXOLVE ® 7 | |

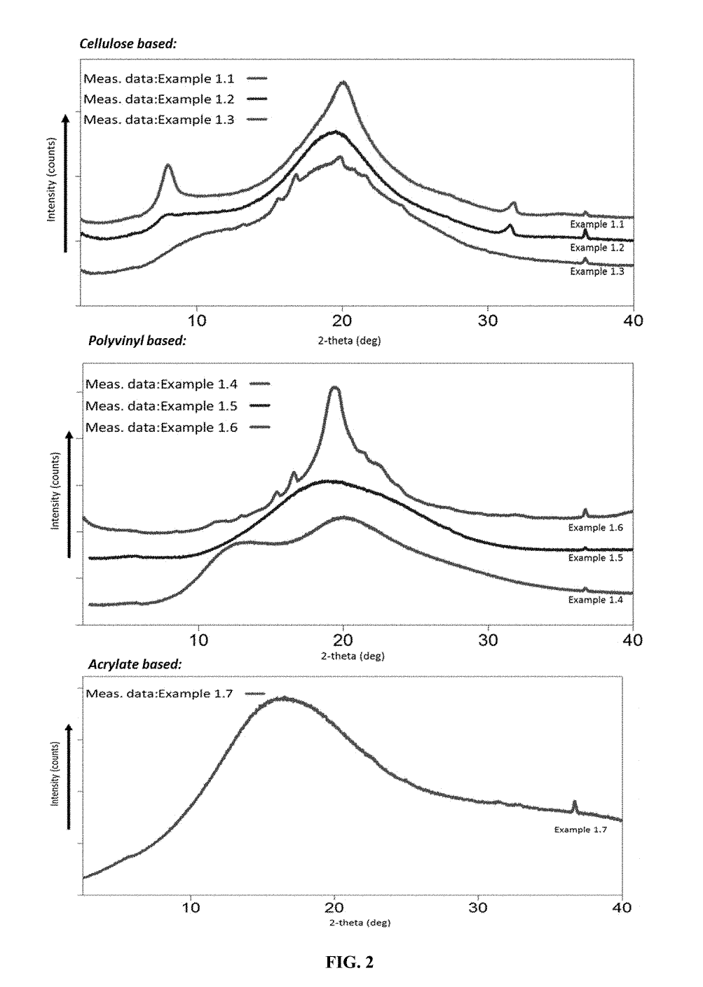

Neat crystalline abiraterone was processable via thermokinetic compounding with hydroxy propyl β cyclodextrin. Binary mixtures of neat crystalline abiraterone and all other cyclodextrins tested remained unprocessed (at the investigated processing conditions), because friction was not sufficient to obtain ejection temperature. The processed mixture was analyzed via XRD as described above in Example 1. The resulting X-ray diffractogram, shown in FIG. 3, confirmed that an amorphous solid dispersion was formed. It is expected that the other cyclodextrins tested may be processable if pre-treated by granulation or slugging, allowing sufficient friction to occur during thermokinetic compounding. Alternatively, processing these mixtures on a manufacturing-scale thermokinetic compounder may provide sufficient friction and shear to yield amorphous compositions that were not possible on the research-scale machine.

### Example 3: Dissolution Testing of Abiraterone Pharmaceutical Formulations

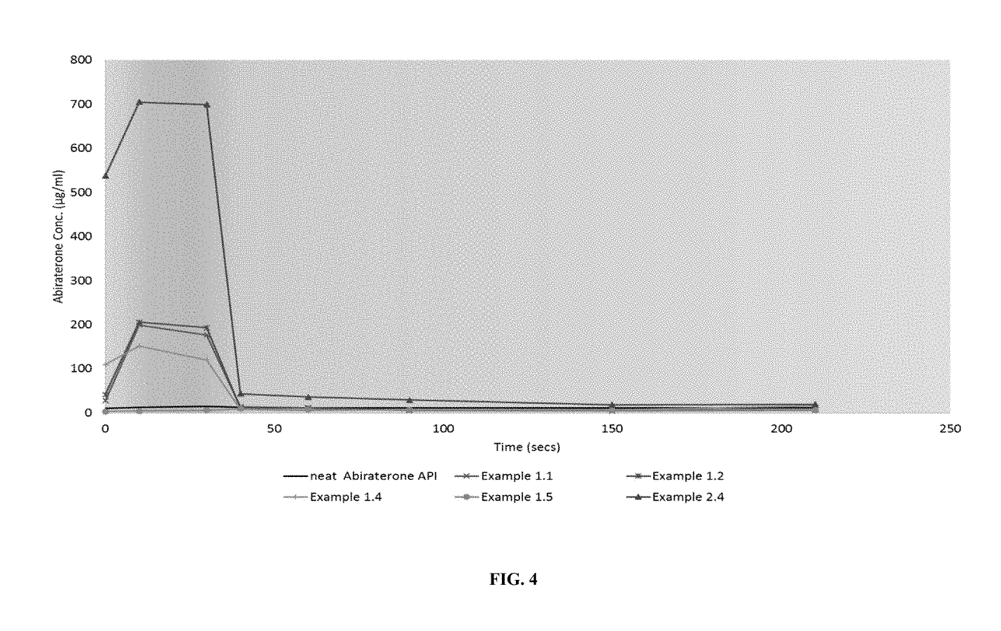

The dissolution performance of the various pharmaceutical formulations of abiraterone or neat crystalline abiraterone was analyzed using a supersaturated, non-sink, bi-phasic dissolution study. Samples equivalent to 31 mg of neat crystalline abiraterone were loaded in a dissolution vessel containing 35 ml of 0.01N HCl and placed in an incubator-shaker set to 37° C. and a rotational speed of 180 rpm. After 30 min, 35 ml of Fasted State Simulated Intestinal Fluid (FaSSIF) was added to the dissolution vessel. At set time points, samples were drawn from the dissolution vessel and centrifuged using an ultracentrifuge. The supernatants were further diluted using a diluent and analyzed by HPLC. Results are presented in FIG. 4.

Almost all of the tested pharmaceutical formulations of abiraterone with a polymer excipient or a cyclic oligomer excipient showed a higher rate and extent of dissolution as compared to neat crystalline abiraterone. Amongst the amorphous pharmaceutical formulations, the one containing a hydroxy propyl β cyclodextrin excipient showed a significantly higher extent of dissolution as compared to the pharmaceutical formulations containing a polymer excipient. This result was quite unexpected because very typically polymers are superior to all other excipients with respect to dissolution performance in ASDs formulations. Hence, it would not be predicted that a non-polymer, in this case a cyclic oligomer, would provide superior abiraterone dissolution performance, and certainly not to the extent shown in FIG. 4.

### Example 4: Dissolution Testing of Abiraterone Pharmaceutical Formulations with Secondary Excipients

Although the hydroxy propyl β cyclodextrin excipient provided enhanced abiraterone dissolution in the acidic phase of dissolution testing, in the neutral phase, the abiraterone precipitated owing to its weakly basic nature and substantially poorer solubility when in the unionized state. Therefore, it was hypothesized that adding a secondary excipient to the formulation could reduce the rate of precipitation in the neutral phase, thus resulting in greater overall solubility enhancement.

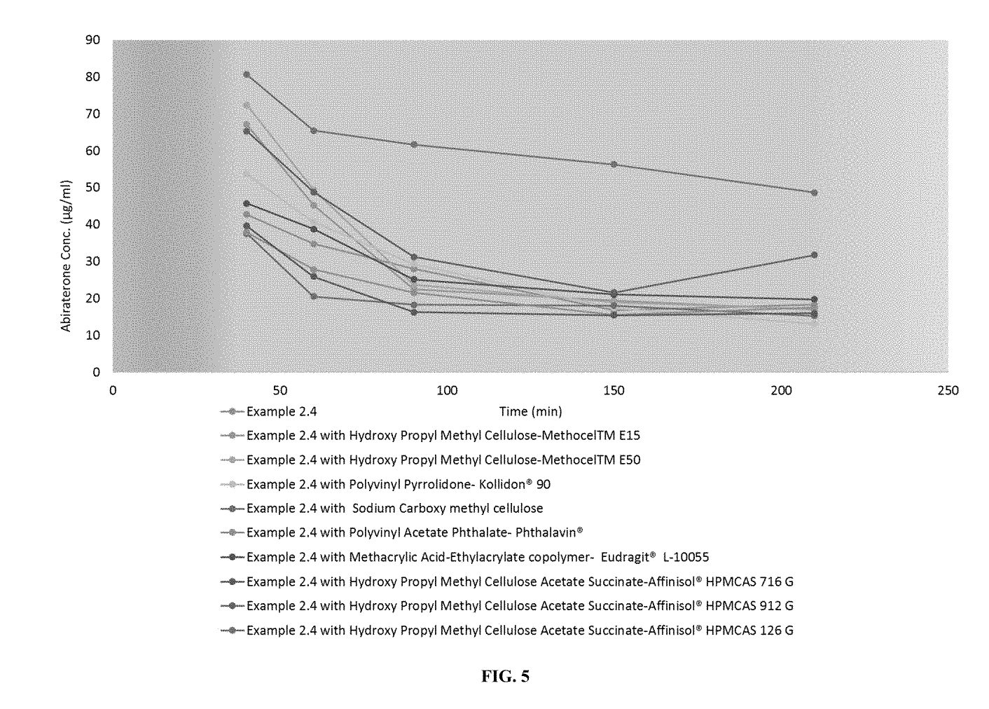

To screen secondary polymer excipients to potentially improve abiraterone dissolution in the neutral phase, an amorphous solid dispersion of 10% by weight abiraterone and 90% by weight hydroxy propyl β cyclodextrin was prepared, and samples were subjected to the acidic phase of dissolution testing using dissolution media containing 35 mg of various secondary polymers. FIG. 5 presents the results of these experiments. All secondary polymer excipients had a slight negative impact on acid phase dissolution, resulting in less than a 20% decrease in area under the dissolution curve for the relevant samples as compared to a sample with no polymer secondary excipients. In the neutral phase of the dissolution test, sodium carboxy methyl cellulose, polyvinyl acetate phthalate and hydroxy propyl methyl cellulose acetate succinate with 5-9% acetate substitution and 14-18% of succinate substitution, all had negative effects on dissolution. However, all remaining secondary excipients showed a positive impact, with hydroxy propyl methyl cellulose acetate succinate with 10-14% acetate substitution and 4-8% of succinate substitution showing the highest positive impact. This secondary polymer excipient caused a 2.4-fold increase in area under the dissolution curve during the neutral phase as compared to a sample with no polymer secondary excipient.

### Example 5: Optimization of Weight Ratios of Abiraterone, Cyclic Oligomer Excipient, and Secondary Excipient in Amorphous Solid Dispersions

Hydroxy propyl β cyclodextrin primary excipient concentration and hydroxy propyl methyl cellulose acetate succinate with 10-14% acetate substitution and 4-8% of succinate substitution secondary excipient concentration were optimized by subjecting various mixtures to thermokinetic compounding. Various KSDs of abiraterone and hydroxy propyl β cyclodextrin primary excipient with various polymer secondary excipients were prepared as in Example 1. Relative weight percentages, excipients, and thermokinetic compounding outcomes are described in Table 3.

TABLE 3
Abiraterone-primary and secondary excipient solid dispersions and thermokinetic
compounding outcomes

| | Composition | | | |
|---|---|---|---|---|
| | | | Secondary | |
| Ex. No. | API (% Wt) | Cyclic Oligomer (% Wt) | Excipient (% Wt) | Outcome |
| 3.1 | Abiraterone (10) | Hydroxy Propyl β | Hydroxy Propyl | Fully |
| | | Cyclodextrin- | Methyl Cellulose | Processed |
| | | KLEPTOSE ® HPB (50) | Acetate Succinate- | |
| | | | AFFINISOL ® | |
| | | | HPMCAS 126 G | |
| | | | (40) | |
| 3.2 | Abiraterone (10) | Hydroxy Propyl β | Hydroxy Propyl | Fully |
| | | Cyclodextrin- | Methyl Cellulose | Processed |
| | | KLEPTOSE ® HPB (60) | Acetate Succinate- | |
| | | | AFFINISOL ® | |
| | | | HPMCAS 126 G | |
| | | | (30) | |
| 3.3 | Abiraterone (10) | Hydroxy Propyl β | Hydroxy Propyl | Fully |
| | | Cyclodextrin- | Methyl Cellulose | Processed |
| | | KLEPTOSE ® HPB (70) | Acetate Succinate- | |
| | | | AFFINISOL ® | |
| | | | HPMCAS 126 G | |
| | | | (20) | |
| 3.4 | Abiraterone (10) | Hydroxy Propyl β | Hydroxy Propyl | Fully |
| | | Cyclodextrin- | Methyl Cellulose | Processed |
| | | KLEPTOSE ® HPB (80) | Acetate Succinate- | |
| | | | AFFINISOL ® | |
| | | | HPMCAS 126 G | |
| | | | (10) | |

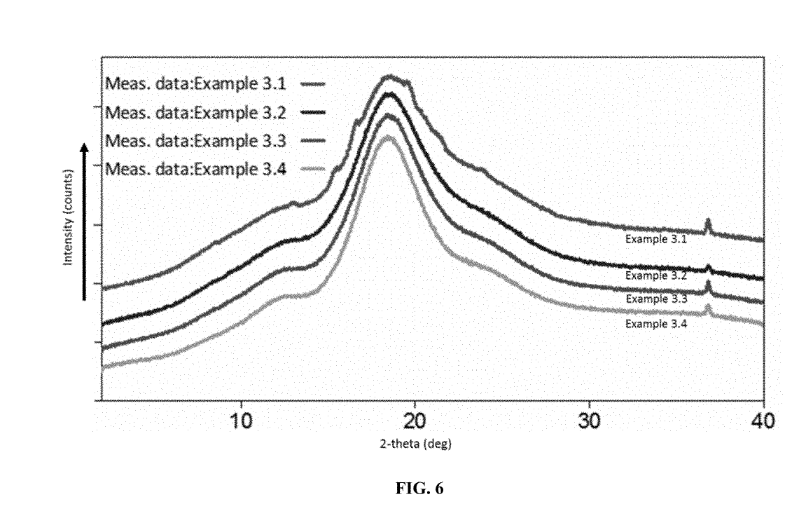

All of the ternary mixtures were processable by thermokinetic compounding. XRD, revealed that Example 3.1, which contained only 50% primary excipient, did not form an amorphous solid dispersion at explored conditions (FIG. 6). The other mixtures did form amorphous solid dispersions (FIG. 6).

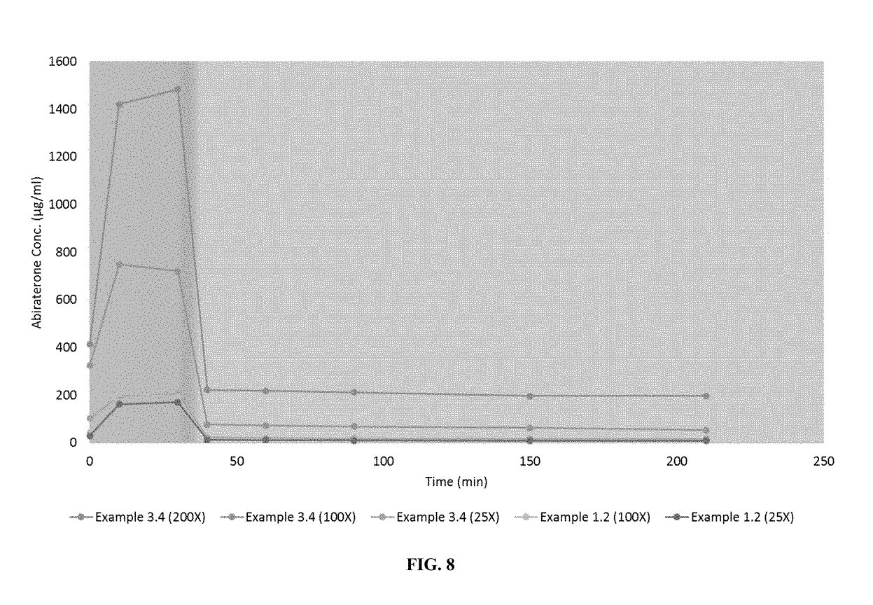

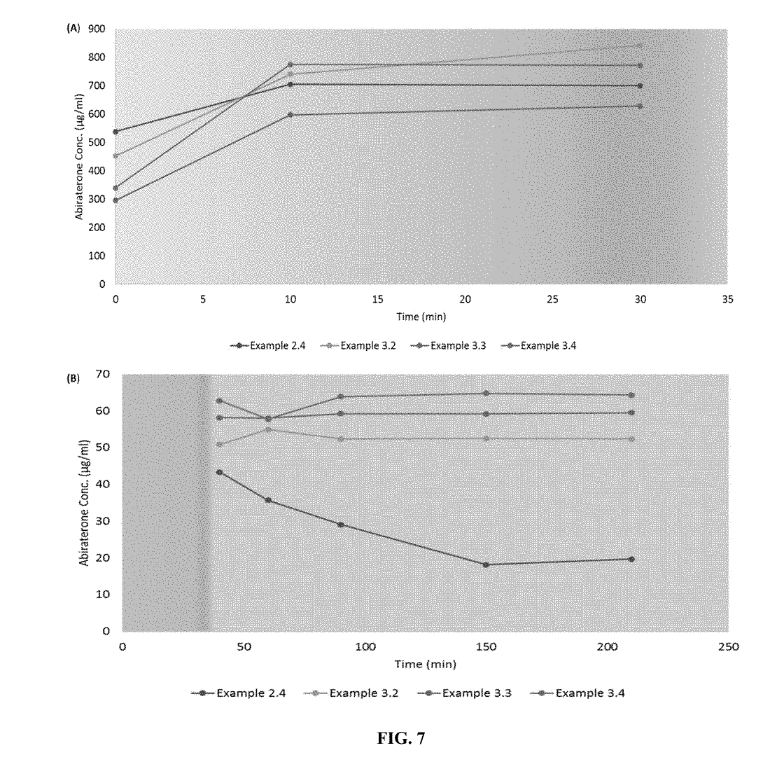

Performance evaluations of all the pharmaceutical compositions including amorphous abiraterone, hydroxy propyl β cyclodextrin and hydroxy propyl methyl cellulose acetate succinate with 10-14% acetate substitution and 4-8% of succinate substitution, were carried out similarly to the dissolution tests in Examples 3 and 4. Results are presented in FIG. 7.

In the acidic dissolution phase, the pharmaceutical formulation of Example 2.4 performed better than all other compositions evaluated. However, in neutral phase, Example 3.4, performed better than the other compositions. Similarly, overall dissolution performance was better for example 3.4 as compared to other compositions.

The results of FIG. 7 also show that, although it might be expected based on the initial tests of Example 4 that higher relative amounts of the polymer secondary excipient in the amorphous solid dispersion would lead to better dissolution enhancement, as the relative amount of polymer secondary excipient is increased, the relative amount of cyclic oligomer primary excipient decreases. This in turn disturbs the molar ratio of abiraterone to cyclic oligomer excipient, which affects dissolution performance.

When amorphous abiraterone in active form and hydroxy propyl β cyclodextrin excipient are present in an amorphous solid dispersion in at weight ratio of 1:9, the molar ratio is 1:2.25. When the weight ratio decreases to 1:8, the molar ratio decreases to 1:2. Up to this point, optimal dissolution is still observed. However, when the molar ratio decreases to below 1:2, it appears that the dissolution enhancement may begin to decline.

### Example 6: Super-Saturation Studies with an Abiraterone-Hydroxy Propyl R Cyclodextrin-Hydroxy Propyl Methyl Cellulose Acetate Succinate with 10-14% Acetate Substitution and 4-8% of Succinate Substitution ASD

Conventionally, in a non-sink, bi-phasic dissolution study, it is expected that a pharmaceutical formulation reaches a certain degree of super-saturation for the API dissolved in the dissolution medium, at which point the addition of more of the pharmaceutical formulation to the dissolution medium does not lead to a further increase in the concentration of the API dissolved in the dissolution medium. This is considered the super-saturation threshold: the maximum amount of API that will dissolve in the dissolution media with that formulation. In order to investigate this phenomenon and determine the super-saturation threshold for abiraterone pharmaceutical formulations of the present disclosure, formulations of Example 3.4 (cyclic oligomer primary excipient with polymer secondary excipient) and Example 1.2 (polymer excipient) were tested at varying amounts. Specifically, formulations resulting in abiraterone levels of 200× (˜62 mg of abiraterone), 100× (˜31 mg of abiraterone) and 25× (˜7.7 mg of abiraterone), as compared to the intrinsic solubility of abiraterone in FaSSIF medium, were prepared. A dissolution study was carried out as in Example 3 and results are presented in FIG. 8.

For pharmaceutical formulations of Example 3.4, it was observed that as the initial loading of the composition increased from 25×, to 100× and further to 200×, the concentration of abiraterone in the dissolution medium in both the acidic phase and neutral phase increased significantly. Conversely, when the pharmaceutical formulation of Example 1.2 was evaluated at levels of 25× and 100×, only a negligible increase in concentration of abiraterone in the dissolution medium was observed. These results demonstrate that an amorphous solid dispersion containing abiraterone with a cyclic oligomer primary excipient and a polymer secondary excipient can provide enhanced dissolution and a substantially greater super-saturation threshold as compared to amorphous solid dispersions with a polymer primary excipient.

A pharmaceutical formulation of the present disclosure may result in at least 100 times, at least 200 times, at least 500 times, or at least 700 times the concentration of neat crystalline abiraterone when a 31 mg equivalent of abiraterone in the active form in the pharmaceutical formulation is added to 35 mL or 0.01N HCl.

### Example 7: Abiraterone-Hydroxy Propyl β Cyclodextrin Pharmaceutical Formulations with Increased Abiraterone Loading

Abiraterone was processed with hydroxy propyl β cyclodextrin in weight ratios of 1:9, 1:4, and 3:7 by thermokinetic compounding and milled per the methods described in Example 1. The formulation details and thermokinetic compounding outcomes are described in Table 4.

TABLE 4
Abiraterone-hydroxy propyl β cyclodextrin solid dispersions of
varying drug loading and thermokinetic compounding outcomes

| | Composition | | Thermo-Kinetic |
|---|---|---|---|
| | | Cyclic Oligomer | Processing |
| Example No. | API (% Wt) | (% Wt) | Outcome |
| 2.4 | Abiraterone (10) | Hydroxy Propyl β | Fully Processed |
| | | Cyclodextrin-Klep- | |
| | | tose ® HPB (90) | |
| 7.1 | Abiraterone (20) | Hydroxy Propyl β | Fully Processed |
| | | Cyclodextrin-Klep- | |
| | | tose ® HPB (80) | |
| 7.2 | Abiraterone (30) | Hydroxy Propyl β | Fully Processed |
| | | Cyclodextrin-Klep- | |
| | | tose ® HPB (70) | |

The processed formulations were analyzed via XRD by the method described in Example 1. The resulting X-ray diffractograms, shown in FIG. 9, confirmed that an amorphous solid dispersion was formed.

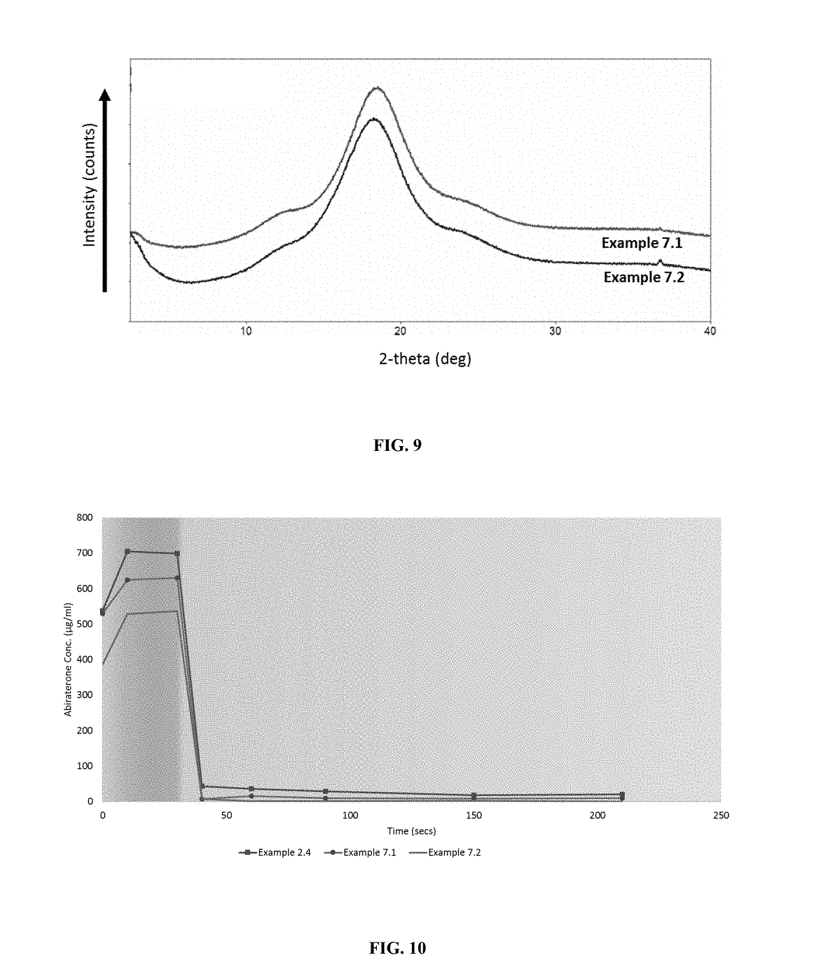

The formulations were then dissolution tested per the method of Example 3. These results are presented in FIG. 10. The dissolution results, for all formulations, show substantially enhanced solubility and dissolution properties relative to crystalline abiraterone. However, the extent of supersaturation was determined to be dependent on the abiraterone-to-hydroxy propyl β cyclodextrin ratio, with the lower ratio resulting in greater dissolution and solubility enhancement. The observation that the 1:9 weight ratio provided the best result by this dissolution test corroborates the discussion from Example 5 and conclusion that the preferred molar ratio of abiraterone-to-hydroxy propyl β cyclodextrin is greater than or equal to about 1:2.

### Example 8: Solid Dispersions of Abiraterone Acetate with Various Polymer Excipients

Abiraterone acetate was processed with various polymers in a 1:9 weight ratio by thermokinetic compounding and milled per the methods described in Example 1. The formulation details and thermokinetic compounding outcomes are described in Table 5.

TABLE 5
Abiraterone acetate-polymer excipient solid dispersions and thermokinetic
compounding outcomes

| | | | Thermo- |
|---|---|---|---|
| | | | kinetic |
| Ex. | Composition | | Processing |
| No. | API (10% Wt) | Polymer (90% Wt) | Outcome |

Cellulose based

| | | | |
|---|---|---|---|
| 8.1 | Abiraterone Acetate | Hydroxy Propyl Methyl | Fully |
| | | Cellulose-Methocel ™ E5 | Processed |
| 8.2 | Abiraterone Acetate | Hydroxy Propyl Methyl | Fully |
| | | Cellulose Acetate Succinate- | Processed |
| | | Affinisol ® HPMCAS 126 G | |
| 8.3 | Abiraterone Acetate | Hydroxy Propyl Methyl | Fully |
| | | Cellulose Phthalate- | Processed |
| | | Hypromellose Phthalate | |

Polyvinyl based

| | | | |
|---|---|---|---|
| 8.4 | Abiraterone Acetate | Polyvinyl Pyrrolidone- | Fully |
| | | Kollidong 30 | Processed |
| 8.5 | Abiraterone Acetate | Vinylpyrrolidone-vinyl acetate | Fully |
| | | copolymer- | Processed |
| | | Kollidon ® VA 64 | |
| 8.6 | Abiraterone Acetate | Polyethylene glycol, polyvinyl | Fully |
| | | acetate and | Processed |
| | | polyvinylcaprolactame-based | |
| | | graft copolymer- | |
| | | Soluplus ® | |

Acrylate based

| | | | |
|---|---|---|---|
| 8.7 | Abiraterone Acetate | An anionic copolymer based on | Fully |
| | | methacrylic acid and ethyl | Processed |
| | | acrylate- | |
| | | Eudragit ® L 100-55 | |

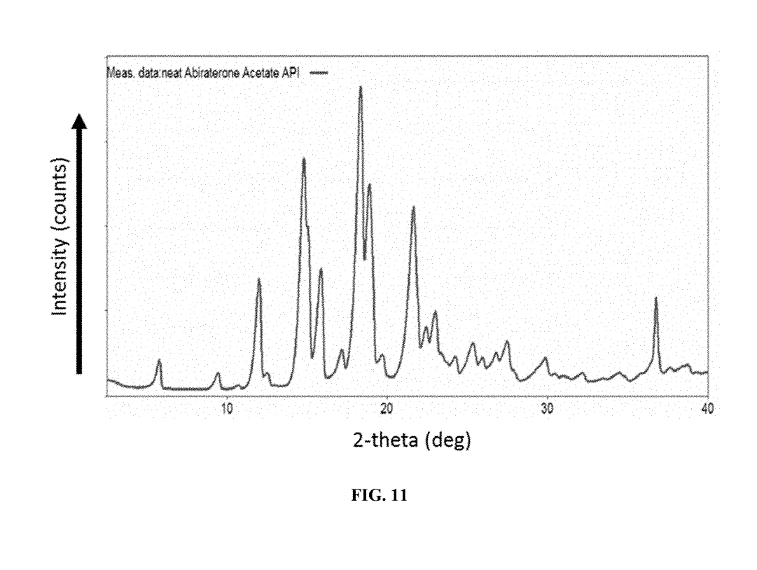

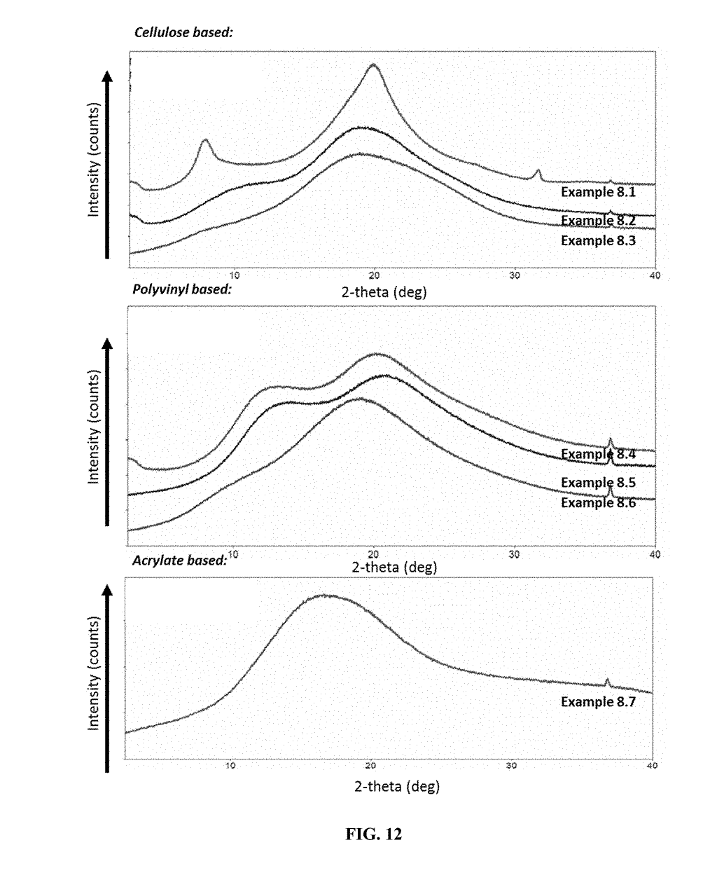

Bulk abiraterone acetate and the processed formulation were analyzed via XRD per the method described in Example 1. The resulting X-ray diffractogram for the drug substance and the processed formulations are shown in FIGS. 11 and 12, respectively. The results shown in FIG. 12 confirmed that amorphous solid dispersions of abiraterone acetate and various polymers were formed by thermokinetic compounding.

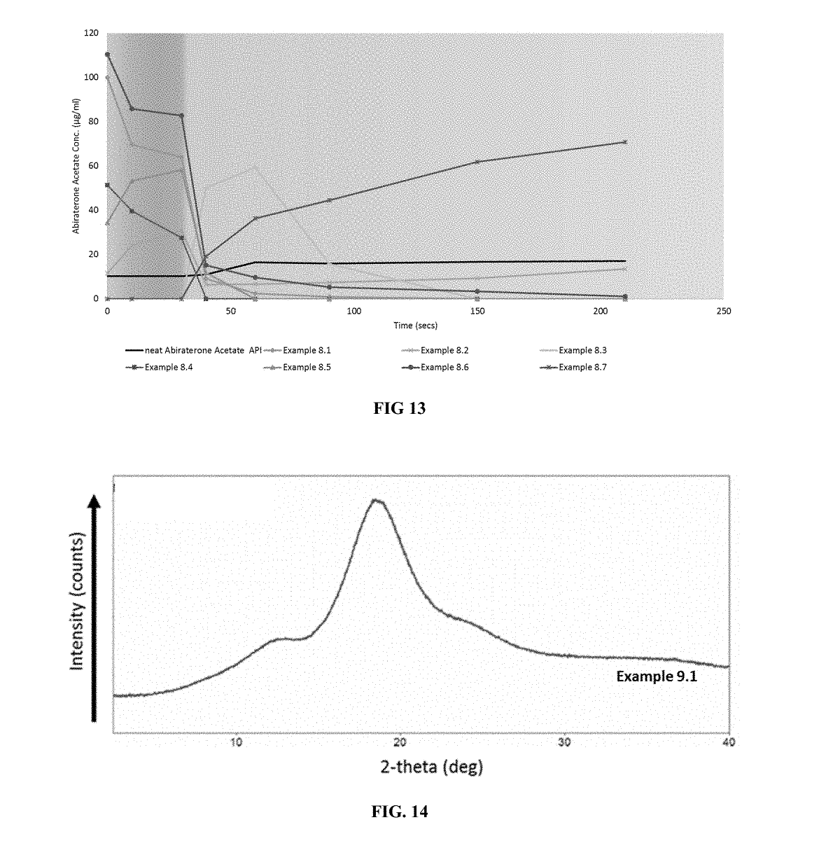

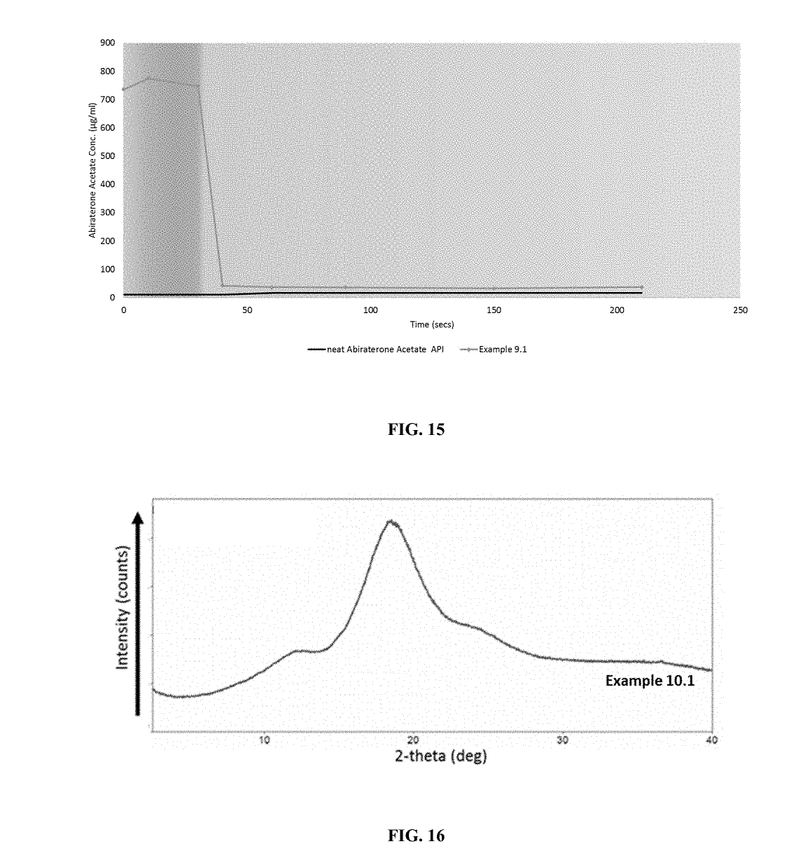

The abiraterone acetate-polymer amorphous dispersions were then dissolution tested against neat abiraterone acetate per the method of Example 3. These results are presented in FIG. 13. The dissolution results demonstrate a improvement in the rate and extent of abiraterone acetate relative to the neat drug. However, these dissolution results are inferior to dissolution results demonstrated by example 9.1 in FIG. 15.

FIG. 13 is a graph of concentration of dissolved abiraterone acetate versus time (dissolution profile) for neat crystalline abiraterone acetate and amorphous solid dispersions of abiraterone acetate with various polymers.

### Example 9: Solid Dispersions of Abiraterone Acetate-Hydroxy Propyl β Cyclodextrin

Abiraterone acetate was processed with hydroxy propyl β cyclodextrin in a 1:9 weight ratio by thermokinetic compounding and milled per the methods described in Example 1. The formulation details and thermokinetic compounding outcomes are described in Table 6.

TABLE 6
Abiraterone acetate-hydroxy propyl β cyclodextrin solid dispersion
composition and thermokinetic compounding outcomes

| | Composition | | Thermo-Kinetic |
|---|---|---|---|
| | | Cyclic Oligomer | Processing Out- |
| Ex. No. | API (% Wt) | (% Wt) | come |

Modified cyclic oligomer

| | | | |
|---|---|---|---|
| 9.1 | Abiraterone Acetate (10) | Hydroxy Propyl β | Processed |
| | | Cyclodextrin- | |
| | | Kleptose ® | |
| | | HPB (90) | |

Bulk abiraterone acetate and the processed formulation were analyzed via XRD per the method described in Example 1. The resulting X-ray diffractogram for the drug substance and the processed formulations are shown in FIGS. 11 and 14, respectively. The result shown in FIG. 14 confirmed that an amorphous solid dispersion of abiraterone acetate and hydroxy propyl β cyclodextrin was formed by thermokinetic compounding.

The abiraterone acetate-hydroxy propyl β cyclodextrin amorphous dispersion was then dissolution tested against neat abiraterone acetate per the method of Example 3. These results are presented in FIG. 15. The dissolution results demonstrate a substantial improvement in the rate and extent of abiraterone acetate dissolution during the acidic phase of the test for the KSD formulation relative to the neat drug. While extensive drug precipitation was observed for the KSD composition upon transition to the neutral phase of the test, the plateau drug concentration remained superior to the crystalline drug control.

Amongst the amorphous pharmaceutical formulations of abiraterone acetate, the one containing a hydroxy propyl β cyclodextrin excipient showed a significantly higher extent of dissolution as compared to the pharmaceutical formulations containing a polymer excipient. This result was quite unexpected because very typically polymers are superior to all other excipients with respect to dissolution performance in ASDs formulations. Hence, it would not be predicted that a non-polymer, in this case a cyclic oligomer, would provide superior abiraterone dissolution performance, and certainly not to the extent shown in FIG. 15.

FIG. 15 is a graph of concentration of dissolved abiraterone acetate versus time (dissolution profile) for neat crystalline abiraterone acetate and an amorphous solid dispersion of abiraterone acetate with hydroxy propyl β cyclodextrin.

### Example 10: Abiraterone Acetate-Hydroxy Propyl β Cyclodextrin Pharmaceutical Formulations with Increased Abiraterone Acetate Loading

Abiraterone acetate was processed with hydroxy propyl β cyclodextrin in weight ratios of 1:9 and 1:4 by thermokinetic compounding and milled per the methods described in Example 1. The formulation details and thermokinetic compounding outcomes are described in Table 7.

TABLE 7
Abiraterone acetate-hydroxy propyl β cyclodextrin solid dispersions
of varying drug loading and thermokinetic compounding outcomes

| | Composition | | Thermo-Kinetic |
|---|---|---|---|
| | | Cyclic Oligomer (% | Processing |
| Example No. | API (% Wt) | Wt) | Outcome |
| 9.1 | Abiraterone | Hydroxy Propyl β | Fully Processed |
| | Acetate (10) | Cyclodextrin-Klep- | |
| | | tose ® HPB (90) | |
| 10.1 | Abiraterone | Hydroxy Propyl β | Fully Processed |
| | Acetate (20) | Cyclodextrin-Klep- | |
| | | tose ® HPB (80) | |

The processed formulations were analyzed via XRD by the method described in Example 1. The resulting X-ray diffractogram, shown in FIG. 16, confirmed that an amorphous solid dispersion was formed at the higher loading of abiraterone acetate.

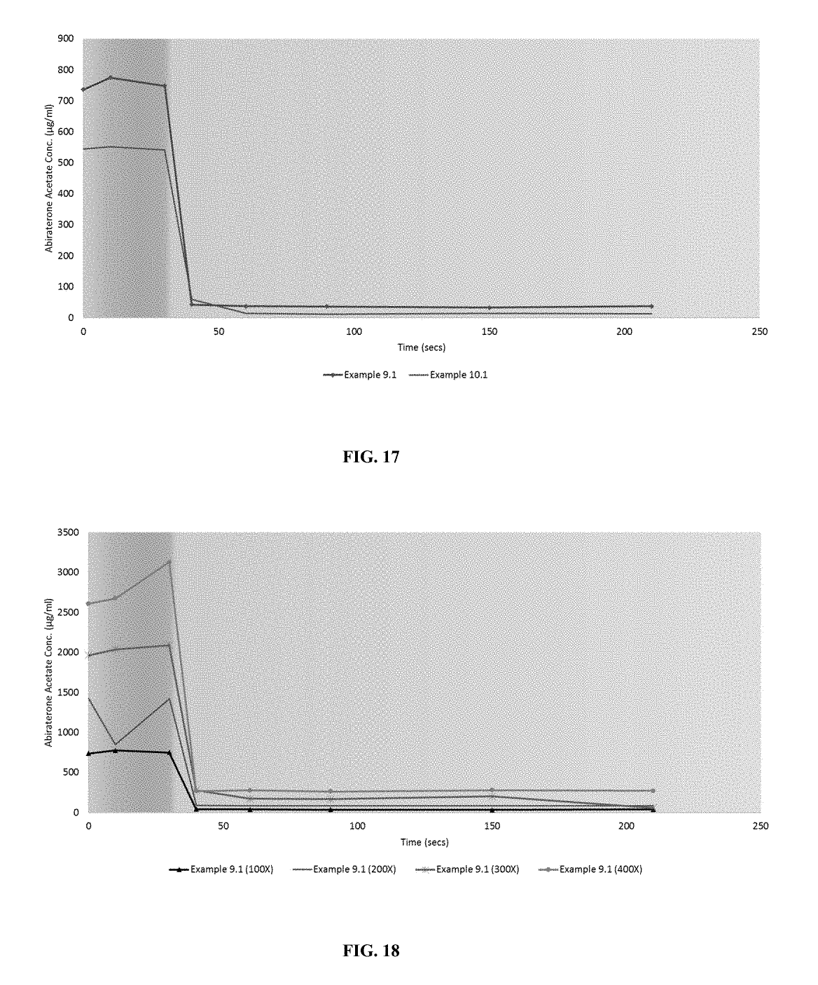

The formulations were then dissolution tested per the method of Example 3. These results are presented in FIG. 17. The dissolution results show that the extent of supersaturation was dependent on the abiraterone acetate-to-hydroxy propyl β cyclodextrin ratio, with the lower ratio resulting in greater dissolution and solubility enhancement. The observation that the 1:9 weight ratio provided the best result by this dissolution test corroborates the discussion from Example 5 and the conclusion that the preferred molar ratio of abiraterone/abiraterone acetate-to-hydroxy propyl β cyclodextrin is greater than or equal to about 1:2.

### Example 11: Super-Saturation Studies with an Abiraterone Acetate-Hydroxy Propyl β Cyclodextrin ASD

Conventionally, in a non-sink, bi-phasic dissolution study, it is expected that a pharmaceutical formulation reaches a certain degree of super-saturation for the API dissolved in the dissolution medium, at which point the addition of more of the pharmaceutical formulation to the dissolution medium does not lead to a further increase in the concentration of the API dissolved in the dissolution medium. This is considered the super-saturation threshold: the maximum amount of API that will dissolve in the dissolution media with that formulation. In order to determine the super-saturation threshold for the abiraterone acetate-hydroxy propyl β cyclodextrin (1:9) ASD of Example 9.1, the formulation was tested at concentrations varying from 400 to 100-times the intrinsic solubility of abiraterone in FaSSIF medium. A dissolution study was carried out as in Example 3 and results are presented in FIG. 18.

For a pharmaceutical formulation of Example 9.1, it was observed that as the initial loading of the composition increased from 100×, to 200×, to 300×, and finally to 400×, the concentration of abiraterone in the dissolution medium in both the acidic phase and neutral phase increased significantly. These results demonstrate that an amorphous solid dispersion containing abiraterone acetate with a cyclic oligomer excipient can provide enhanced dissolution and a substantially improved super-saturation threshold as compared to the neat drug substance.

### Example 11: Development of Immediate Release and Gastro-Retentive/Extended Release Tablets Containing ASDs of Abiraterone with Hydroxy Propyl β Cyclodextrin

In the design of a final dosage forms containing the abiraterone-cyclic oligomer amorphous solid dispersions of this disclosure, it was desired to have tablets of varying drug release rates to enable different pharmacokinetic profiles that could have unique therapeutic benefits. Therefore, an immediate release (IR) tablet was developed along with a gastro-retentive extended release (XR) tablet. Example compositions of both are provided in Table 8.

TABLE 8
Development of immediate release and gastro-retentive/extended release tablets
containing an abiraterone-hydroxy propyl β cyclodextrin amorphous solid dispersion

| | | | |
|---|---|---|---|
| | | | Example 11.2 |
| | | Example 11.1 | Gastro-retentive/ |
| | | Immediate Release | Modified/Sustained |
| Component and Quality Standard (and | | Tablet | Release Tablet |
| Grade, if applicable) | Function | % (w/w) | % (w/w) |

Drug Product Intermediate (Example 2.4)

| | | | |
|---|---|---|---|
| Abiraterone | Active Ingredient | 5.00 | 5.00 |
| Hydroxy Propyl β Cyclodextrin (Kleptose | Stabilizing | 45.00 | 45.00 |
| HPB) | diluent/solubilizer | | |

External Phase Excipients

| | | | |
|---|---|---|---|
| Microcrystalline cellulose (Avicel PH-102) | Diluent/ | 24.10 | 5.61 |
| | binder | | |
| Hydroxy Propyl β Cyclodextrin (Klep- | Solubilizer | 5.60 | — |
| tose ® HPB) | | | |
| HPMCAS HMP grade (AQOAT ®) | Solubilizer | 3.93 | — |
| Polyethylene Oxide(Polyox WSR 301) | Controlled | — | 24.33 |
| | release | | |
| | agent | | |
| Hydroxy Propyl Methyl Cellulose (Methocel | Controlled | — | 17.93 |
| E4M) | release | | |
| | agent | | |
| Mannitol (Pearlitol 200SD) | Diluent/ | 10.37 | 1.13 |
| | binder | | |
| Croscarmallose Na (VIVASOL ®) | Disintegrant | 5.00 | 0.00 |
| Colloidal Silicon Dioxide (Aerosil 200) | Glidant | 0.50 | 0.50 |
| Magnesium Stearate | Lubricant | 0.50 | 0.50 |
| Total | | 100.00 | 100.00 |

The compositions shown in Table 8 were produced by blending the abiraterone-hydroxy propyl β cyclodextrin ASD powder with the tableting excipients in a suitable powder blender, then directly compressing this blend to a desired hardness with a suitable pharmaceutical tablet press.

In the case of the IR tablet, the external phase is conventional to a disintegration tablet with the exception of HPMCAS and hydroxy propyl β cyclodextrin, which are included to promote abiraterone supersaturation, particularly in the intestinal lumen.

In the case of the XR tablet, the external phase contains the functional polymers, polyethylene oxide and hydroxypropylmethyl cellulose. These polymers are incorporated into the external phase as gelling agents to promote swelling of the tablet in the stomach to: (1) facilitate retention of the tablet in the stomach and (2) modify the release of the solubility enhanced ASD form of abiraterone. This tablet design is intended to sequester the abiraterone dose in the acidic environment of the stomach where the drug is more soluble and prolong release of dissolved abiraterone in the intestinal tract such that consistent, therapeutic abiraterone exposure is achieved for the duration of therapy.

### Example 12: Dissolution Testing of Tablets Produced Per Example 11

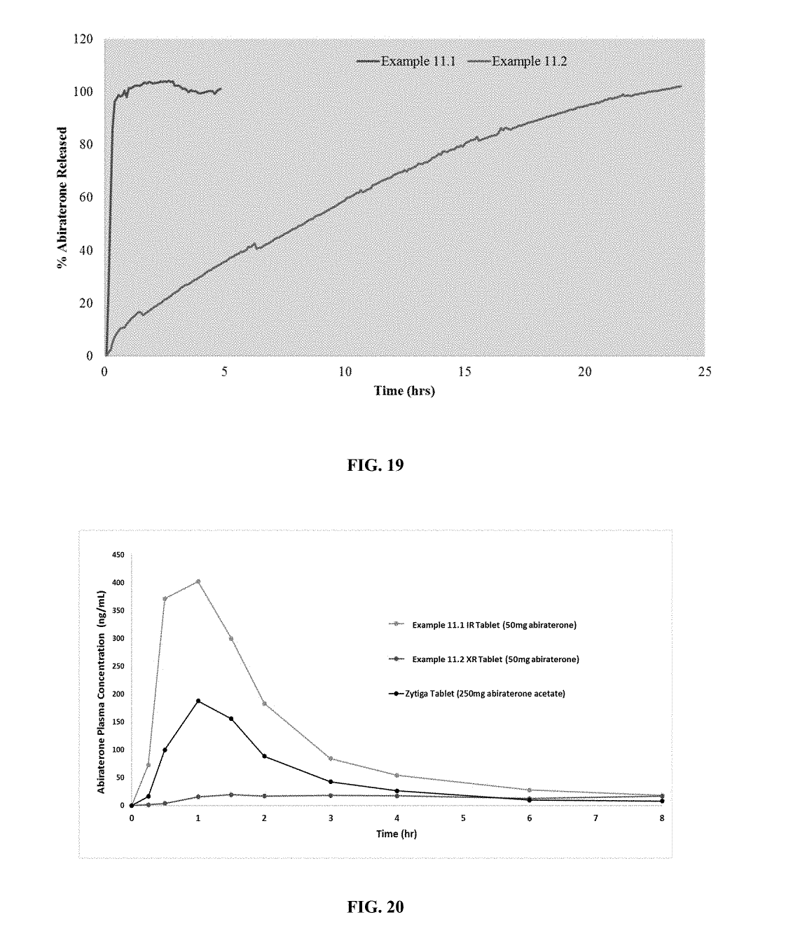

The tablets made according to Example 11 were dissolution tested to determine the rate of abiraterone release from the IR and XR dosage forms. A USP apparatus II (paddle) dissolution tester equipped with fiber-optic UV-spectroscopy for in-situ drug concentration measurements was used to conduct the analysis. Tablets of 50 mg strength were placed in dissolution vessels containing 900 ml of 0.01 N HCl heated to approximately 37° C. with a paddle stirring rate of 75 RPM. The results of this test are presented in FIG. 19.

The dissolution results shown in FIG. 19 demonstrate the rapid and complete release of abiraterone from the IR tablet of Example 11.1 and the prolonged abiraterone release over 24 hours for the XR tablet of Example 11.2. When administered to patients it is expected that the IR tablet will result in rapid and complete absorption with a high Cₘₐₓ-to-Cₘᵢₙ ratio. Whereas, the XR tablet will result in prolonged absorption resulting in a reduced Cₘₐₓ-to-Cₘᵢₙ ratio relative to the IR tablet and the current commercial products, i.e., Zytiga and Yonsa. This reduced Cₘₐₓ-to-Cₘᵢₙ ratio may provide therapeutic benefit in cases were maintenance of abiraterone concentrations within the therapeutic window for the duration of treatment is critical to the therapeutic outcome. In these cases, the fast absorption and elimination of an immediate release dosage forms is undesirable because abiraterone plasma concentrations fall below the therapeutic threshold for some period of time prior to the next dose, which may promote disease progression.

### Example 13: Pharmacokinetic Testing in Male Beagle Dogs of Tablets Made Per Example 11

To evaluate the in vivo performance of the IR and XR tablets presented in Example 10, the tablets (50 mg abiraterone) were orally administered to male beagle dogs along with Zytiga (250 mg abiraterone acetate) in a three-way crossover study design. Study dogs were assigned to dosing groups as shown in the Table 9. The animals received the test articles as a single oral dose. The tablet was placed on the back of the tongue, and the throat was massaged to facilitate swallowing. Then, 10-25 mL of sterile water was administered immediately via syringe to ensure the tablet was washed down into the stomach/swallowed. The first day of dose administration was designated as Day 1 of the study. For all dose events, the animals were fasted overnight and offered food at 4 hours post-dose (after the 4-hour blood collection). There was a 7-day washout between dose events.

TABLE 9
Study parameters for the pharmacokinetic evaluation of abiraterone tablets in
male beagle dogs

| | | | | Amount | | Dosing |
|---|---|---|---|---|---|---|
| Dose | Number of | Dose | Test | Dosed | | Fed/Fasted |
| Group | Animals | Event | Article | (mg) | Purpose | State |
| 1 | 5 | 1 | Example 11.1 (Abiraterone) | 50 | Test Article | Fasted |
| | | | IR Tablets | | | |
| 1 | 5 | 2 | Example 11.2 (Abiraterone) | 50 | Test Article | Fasted |
| | | | XR Tablets | | | |
| 1 | 5 | 3 | Zytiga (Abiraterone Acetate) | 250 | Reference | Fasted |
| | | | Tablets | | Test Article | |

Pharmacokinetic (PK) analysis was performed comparing the IR and XR tablets to the Zytiga reference tablet. The PK parameters are presented in Table 10 and the plasma concentration versus time profiles are provided in FIG. 20. PK analysis comparing abiraterone IR tablets of Example 11.1 to Zytiga established the geometric mean ratios of dose-normalized AUC₀₋₈ and Cₘₐₓ to be 14.7 and 13.9, respectively. These values indicate that the total oral exposure of abiraterone following oral administration of abiraterone IR tablets is approximately 15-fold greater than Zytiga with plasma concentrations at peak being approximately 14-fold greater. This result signifies a substantial improvement in the bioavailability of abiraterone generated by a composition of the current invention over the commercial product, Zytiga. To the inventors' knowledge, such high plasma concentrations relative to dose, as seen with the IR tablet of Example 11.1, have not been previously reported in the literature, and thus signify the uniqueness of this composition.

PK analysis comparing abiraterone XR tablets of Example 11.2 to Zytiga established the geometric mean ratios of dose-normalized AUC₀₋₈ and Cₘₐₓ to be 1.8 and 0.79, respectively. These values indicate that the XR tablet approximately doubled total exposure (AUC) while reducing peak abiraterone plasma concentrations (Cₘₐₓ), hence decreasing the Cₘₐₓ-to-Cₘᵢₙ ratio relative to Zytiga. Given the extreme solubility challenges presented by abiraterone, particularly in the neutral pH of the intestinal lumen, such a result has not been previously achieved. It is only through the unique combination of the novel, solubility enhanced abiraterone-cyclic oligomer ASD with the hydrogel matrix of the XR tablet of this invention that such a result could be realized. Once again, the unique PK profile brought about by the drug release profile of the abiraterone XR tablet is expected to provide therapeutic benefit in cases where consistent, round-the-clock drug levels beyond the therapeutic threshold are required to achieve the desired medical outcome.

TABLE 10
Pharmacokinetic summary of Abiraterone IR and XR Tablets (50 mg
abiraterone) versus Zytiga (250 mg abiraterone acetate) in fasted male beagle dogs
following administration of a single oral dose.

| | | | Dose-Normalized | Dose-Normalized | Dose-Normalized |
|---|---|---|---|---|---|
| | Tₘₐₓ | T_{1/2} | Cₘₐₓ | AUC₀₋₈ | AUCₗₐₛₜ |
| Test Article | (hr) | (hr) | (kg * ng/mL/mg) | (hr * kg * ng/mL/mg) | (hr * kg * ng/mL/mg) |
| Example 11.1 | 0.90 ± 0.42 | 5.51 ± 1.33 | 84.8 ± 17.4 | 156 ± 31.6 | 189 ± 39.9 |
| (Abiraterone) | | | 83.5 (Geo Mean) | 153 (Geo Mean) | 185 (Geo Mean) |
| IR Tablets | | | | | |
| Example 11.2 | 7.20 ± 7.53 | 3.42 ± 0.36 | 8.55 ± 9.81 | 18.9 ± 29.0 | 62.9 ± 77.1 |
| (Abiraterone) | | | 4.71 (Geo Mean) | 9.29 (Geo Mean) | 28.5 (Geo Mean) |
| GR/MR/SR/XR | | | | | |
| Tablets | | | | | |
| Zytiga | 13.35 ± 7.84 | 4.73 ± 0.51 | 30.9 ± 28.7 | 14.1 ± 12.7 | 201 ± 102 |
| (Abiraterone | | | 22.6 (Geo Mean) | 10.4 (Geo Mean) | 179 (Geo Mean) |
| Acetate) | | | | | |
| Tablets | | | | | |

¹Average body weight adjusted dose
²Geometric mean values

### Example 14: Elevated Systemic Concentrations Generated by Abiraterone-Cyclic Oligomer Amorphous Solid Dispersions Lead to Enhanced Tumor Regression in Xenograft Mice

To test the hypothesis that increased systemic concentrations of abiraterone results in improved tumor response, a study was conducted evaluating the efficacy of a composition made according to Example 2.4 relative to abiraterone acetate in a 22RV1 human prostate tumor xenograft model. However, prior to dosing the xenograft mice, an ascending dose PK study was conducted in non-tumored SCID mice to generate the exposure-to-dose curve of the Example 2.4 composition versus abiraterone acetate. Based on this curve, doses were selected for the xenograft study according to the observed systemic exposures.

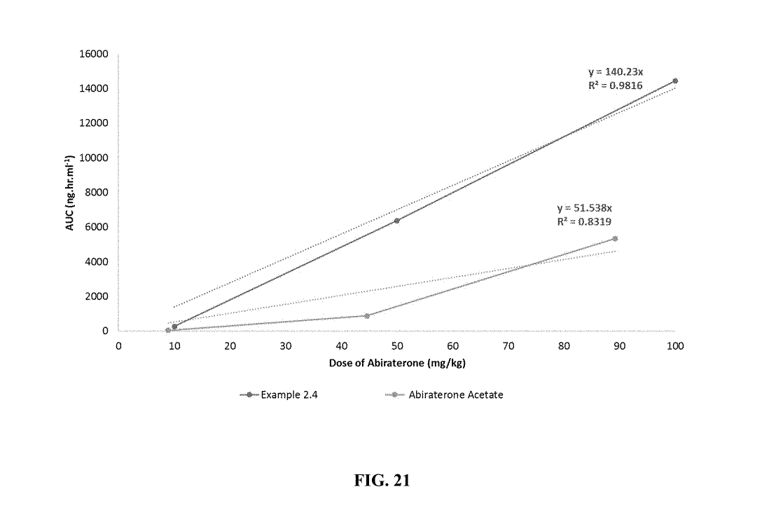

For the PK study, both test articles were dosed by oral gavage as reconstituted powders in an aqueous suspension vehicle. All animals were fasted overnight prior to dosing. The study parameters are summarized in Table 11 and the resulting exposure versus dose curve is presented in FIG. 21.

TABLET 11
Study parameters from the ascending dose study in SCID mice comparing the
pharmacokinetics of Example 2.4 to abiraterone acetate.

| | Number of | | Dose Level | Dose Concentration | Dose Volume | |
|---|---|---|---|---|---|---|
| Group | Males | Abiraterone Formulation | (mg/kg) | (mg/mL) | (mL/kg) | Route |
| 1 | 24 | Example 2.4 | 10 | 1 | 10 | PO |
| 2 | 24 | | 50 | 5 | | |
| 3 | 24 | | 100 | 10 | | |
| 4 | 24 | Abiraterone Acetate | 10 | 1 | | |
| 5 | 24 | | 50 | 5 | | |
| 6 | 24 | | 100 | 10 | | |

The dose-exposure curve shown in FIG. 20 reveals that the dose linearity and total exposure achieved with the Example 2.4 composition is superior to abiraterone acetate. Specifically, the AUC ratio of Example 2.4 to abiraterone acetate at the low, middle, and high doses were 4.0, 7.23, and 2.6, respectively. A linear trendline was fit to both exposure versus dose curves in order to calculate the appropriate xenograft study doses based upon patient exposure data taken from the Zytiga label. From this analysis, low and high doses of abiraterone acetate were determined to be 22.4 and 100 mg/kg, and the corresponding doses for the Example 2.4 composition were 20 mg/kg and 89.2 mg/kg.

The objective of the xenograft mice study was to determine the anti-tumor activity of a composition made per Example 2.4 as a single agent versus abiraterone acetate in the 22RV1 human prostate tumor xenograft model. The study was conducted in CB.17 SCID mice injected with 22RV1 cells (5×10⁶ cells/mouse) in the subcutaneous right flank. Tumors were grown to a mean tumor size between 100 and 150 mm³ prior to study enrollment. The mice were dosed with the test article and reference once-daily by oral gavage per Table 12. Tumor volume was measured throughout the study. The study was terminated on day 26 when mean tumor volume of two experimental groups reached ≥1500 mm³.

TABLE 12
Dosing parameters of the anti-tumor study in 22RV1 xenograft mice

| | | | Abiraterone | |
|---|---|---|---|---|
| | | Vehicle | Acetate | DST-297 |
| | | Control | (QD to | (QD to |
| Group | N | (QD to End) | End) | End) |
| 1. Vehicle Control (PO) | 10 | X | | |
| 2. Abiraterone Acetate | 10 | | X | |
| Dose #1 22.4 mg/kg (PO) | | | | |
| 3. Abiraterone Acetate | 10 | | X | |
| Dose #2 100 mg/kg (PO) | | | | |
| 4. Example 2.4 Dose #1 | 10 | | | X |
| 20 mg/kg (PO) | | | | |
| 5. Example 2.4 Dose #2 | 10 | | | X |
| 89.2 mg/kg (PO) | | | | |

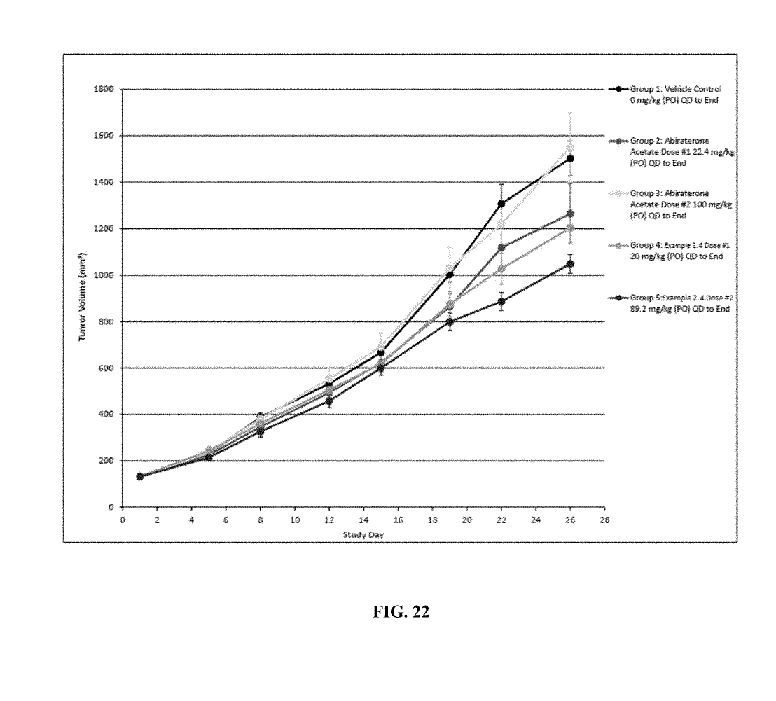

The study results are provided in FIG. 22 and Table 13. The results show that treatment with the Example 2.4 composition showed statistically significant reductions in tumor growth relative to vehicle control at the low (p=0.014) and high (p<0.001) doses. Conversely, treatment with abiraterone acetate did not result in statistically different tumor growth relative to vehicle control. These results clearly demonstrate that the increased systemic abiraterone concentrations achieved by the compositions disclosed herein lead to superior anti-tumor response relative to abiraterone acetate. The in vivo systemic abiraterone exposures observed following oral administration of compositions disclosed by this invention (on a per dose basis) are believed to be the highest published to date; therefore, the Inventors believe this anti-tumor response to be unprecedented. Extrapolating from this result to human patients gives indication that the compositions of the current invention could provide superior therapeutic efficacy to patients with cancers that respond to androgen suppression, such as, prostate and breast cancers.

TABLE 13
Tumor growth resutls following once-daily administration of abiraterone acetate
or the composition from Example 2.4 at two dose levels to 22RV1 xenograft mice.

| | | | | | Tumor Volume (mm³) | | | | |
|---|---|---|---|---|---|---|---|---|---|
| Group | Compound | Dosage | Frequency | Dose Route | Day 26 | | | | |
| 1 | Vehicle Control | 0 mg/kg | QD to End | Oral Gavage | 1,219 | 1,230 | 1,656 | 1,305 | 1,920 |
| 2 | Abiraterone Acetate Dose at #1 | 22.4 mg/kg | QD to End | Oral Gavage | 726 | 1,606 | 1,119 | 1,127 | 1,143 |
| 3 | Abiraterone Acetate Dose at #2 | 100 mg/kg | QD to End | Oral Gavage | 1,609 | 1,639 | 1,084 | 1,490 | 1,429 |
| 4 | Example 2.4 Dose # 1 | 20 mg/kg | QD to End | Oral Gavage | 1,382 | 1,240 | 1,318 | 1,648 | 1,207 |
| 5 | Example 2.4 Dose # 2 | 89.2 mg/kg | QD to End | Oral Gavage | 1,227 | 1,194 | 1,041 | 882 | 1,024 |

| | | | | | | Mean | | Median | | | |
|---|---|---|---|---|---|---|---|---|---|---|---|
| | | | | | | Tumor | | Tumor | | | |
| | Tumor Volume (mm³) | | | | | Volume | | Volume | | Standard | Student's |
| Group | Day 26 | | | | | (mm³) | % T/C | (mm³) | % T/C | Error | t-test |
| 1 | 1,563 | 1,272 | 1,554 | 1,724 | 1,581 | 1502.6 | | 1558.5 | | 75.00 | |
| 2 | 1,062 | 1,389 | 808 | 1,555 | 2,107 | 1264.2 | 84.1% | 1135.0 | 72.8% | 129.95 | 0.165 |
| 3 | 1,453 | 1,152 | 1,568 | 1,287 | 2,787 | 1549.4 | 103.1% | 1471.5 | 94.4% | 149.64 | 0.757 |
| 4 | 979 | 1,133 | 958 | 980 | 1,193 | 1203.8 | 80.1% | 1199.8 | 77.0% | 67.51 | 0.014 |
| 5 | 1,039 | 1,003 | 1,207 | 840 | 1,027 | 1048.4 | 69.8% | 1032.9 | 66.3% | 41.16 | p < 0.001 |

The above disclosure contains various examples of pharmaceutical formulations, final solid dosage forms, methods of forming pharmaceutical formulations, and methods of administering pharmaceutical formulations. Aspects of these various examples may all be combined with one another, even if not expressly combined in the present disclosure, unless they are clearly mutually exclusive. For example, a specific pharmaceutical formulation may contain amounts of components identified more generally or may be administered in any way described herein.

In addition, various example materials are discussed herein and are identified as examples, as suitable materials, and as materials included within a more generally-described type of material, for example by use of the term “including” or “such-as.” All such terms are used without limitation, such that other materials falling within the same general type exemplified but not expressly identified may be used in the present disclosure as well.

The above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A pharmaceutical formulation comprising:
abiraterone; and
a cyclic oligomer excipient.

2. The pharmaceutical formulation of claim 1, wherein the abiraterone and cyclic oligomer excipient are in an amorphous solid dispersion.

3. The pharmaceutical formulation of claim 2, wherein the amorphous solid dispersion contains less than 5% crystalline material.

4. The pharmaceutical formulation of claim 1, wherein the abiraterone comprises at least 99% abiraterone.

5. The pharmaceutical formulation of claim 1, wherein the abiraterone comprises at least 99% abiraterone, having the structural formula:

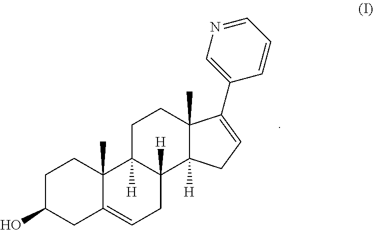

6. The pharmaceutical formulation of claim 1, wherein the abiraterone comprises at least 99% abiraterone salt.

7. The pharmaceutical formulation of claim 1, wherein the abiraterone comprises at least 99% abiraterone ester.

8. The pharmaceutical formulation of claim 7, wherein the abiraterone ester comprises abiraterone acetate, having the structural formula:

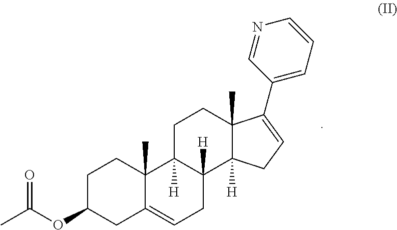

9. The pharmaceutical formulation of claim 1, wherein the abiraterone comprises at least 99% abiraterone solvate.

10. The pharmaceutical formulation of claim 1, wherein the abiraterone comprises at least 99% abiraterone hydrate.

11. The pharmaceutical formulation of claim 1, comprising 10 mg, 25 mg, 50 mg, 70 mg, 75 mg, 100 mg, or 125 mg of amorphous abiraterone.

12. The pharmaceutical formulation of claim 1, comprising an amount of amorphous abiraterone sufficient to achieve the same or greater therapeutic effect, bioavailability, Cₘᵢₙ, Cₘₐₓ or Tₘₐₓ in a patient as 250 mg, 500 mg or 1000 mg of crystalline abiraterone or crystalline abiraterone acetate when consumed on an empty stomach.

13. The pharmaceutical formulation of claim 1, comprising 50 mg of amorphous abiraterone.

14. The pharmaceutical formulation of claim 1, comprising an amount of amorphous abiraterone sufficient to achieve the same or greater therapeutic effect, bioavailability, Cₘᵢₙ, Cₘₐₓ or Tₘₐₓ in a patient as 500 mg of crystalline abiraterone or crystalline abiraterone acetate when consumed on an empty stomach.

15. The pharmaceutical formulation of claim 1, comprising 50 mg or 70 mg of amorphous abiraterone.

16. The pharmaceutical formulation of claim 1, comprising an amount of amorphous abiraterone sufficient to achieve the same or greater therapeutic effect, bioavailability, Cₘᵢₙ, Cₘₐₓ or Tₘₐₓ in a patient as 500 mg or 1,000 mg of crystalline abiraterone or crystalline abiraterone acetate when consumed on an empty stomach.

17. The pharmaceutical formulation of claim 1, wherein the abiraterone and cyclic oligomer are present in a molar ratio of 1:0.25 to 1:25.

18. The pharmaceutical formulation of claim 1, wherein the abiraterone and cyclic oligomer are present in a molar ratio of at least 1:2.

19. The pharmaceutical formulation of claim 1, wherein the amorphous solid dispersion comprises 1% to 50% by weight abiraterone.

20. The pharmaceutical formulation of claim 1, wherein the amorphous solid dispersion comprises at least 10% by weight abiraterone.

21. The pharmaceutical formulation of claim 1, wherein the cyclic oligomer excipient comprises a cyclic oligosaccharide or cyclic oligosaccharide derivative.

22. (canceled)

23. The pharmaceutical formulation of claim 21, wherein the cyclic oligosaccharide or cyclic oligosaccharide derivative comprises a hydroxy propyl β cyclodextrin.

24. The pharmaceutical formulation of claim 21, wherein the cyclic oligosaccharide or cyclic oligosaccharide derivative comprises a sodium (Na) sulfo-butyl ether β cyclodextrin.

25-26. (canceled)

27. The pharmaceutical formulation of claim 1, wherein the amorphous solid dispersion comprises 50% to 99% by weight cyclic oligomer excipient.

28. The pharmaceutical formulation of claim 1, wherein the amorphous solid dispersion comprises at least 60% by weight cyclic oligomer excipient.

29. The pharmaceutical formulation of claim 1, wherein the amorphous solid dispersion comprises an additional excipient.

30-42. (canceled)

43. The pharmaceutical formulation of claim 1, further comprising a glucocorticoid replacement API.

44. (canceled)

45. The pharmaceutical formulation of claim 1, formulated as tablet for oral administration.

46-48. (canceled)

49. The pharmaceutical formulation of claim 45, wherein
the tablet comprises an external phase comprising an additional amount of the cyclic oligomer excipient.

50. (canceled)

51. The pharmaceutical formulation of claim 45, wherein the tablet comprises a concentration enhancing polymer.

52. The pharmaceutical formulation of claim 51, wherein the concentration enhancing polymer comprises a hydroxypropylmethyl cellulose acetate succinate.

53. The pharmaceutical formulation of claim 45, wherein the tablet comprises an external phase comprising at least one additional drug release modifying excipient.

54-55. (canceled)

56. A method of forming a pharmaceutical formulation, the method comprising compounding crystalline abiraterone and a cyclic oligomer excipient in a thermokinetic mixer at a temperature less than or equal to 200° C. for less than 300 seconds to form an amorphous solid dispersion of abiraterone and cyclic oligomer excipient.

57-61. (canceled)

62. A method of forming a pharmaceutical formulation, the method comprising melt processing crystalline abiraterone and a cyclic oligomer excipient to form an amorphous solid dispersion of abiraterone and the cyclic oligomer excipient in which the abiraterone is not substantially thermally degraded.

63-66. (canceled)

67. A method of forming a pharmaceutical formulation, the method comprising dissolving crystalline abiraterone and a cyclic oligomer excipient in a common organic solvent to form a dissolved mixture and spray drying the dissolved mixture to form an amorphous solid dispersion of abiraterone and cyclic oligomer excipient.

68-72. (canceled)

73. A method of treating prostate cancer, breast cancer, salivary cancer or an androgen sensitive cancer in a patient, the method comprising administering a pharmaceutical formulation of claim 1 to a patient having prostate cancer, breast cancer, salivary cancer or an androgen sensitive cancer.

74-115. (canceled)
